(19) United States
(12) Patent Application Publication
(10) Pub. No.: US 2008/0097385 A1
(43) Pub. Date: Apr. 24, 2008

(54) Therapeutic Adjuncts to Enhance the Organ Protective Effects of Postconditioning

(76) Inventors: Jakob Vinten-Johansen, Grayson, GA (US); Zhi-Qing Zhao, Norcross, GA (US)

Correspondence Address: NEEDLE & ROSENBERG, P.C., SUITE 1000, 999 PEACHTREE STREET, ATLANTA, GA, 30309-3915, US

(21) Appl. No.: 11/793,508

(22) PCT Filed: Dec. 20, 2005
(86) PCT No.: PCT/US05/46417

§ 371 Date: Oct. 22, 2007

Related U.S. Application Data
(60) Provisional application No. 60/638,461, filed on Dec. 22, 2004.

(51) Int. Cl. A61M 25/10; A61K 31/165; A61P 43/00

(52) U.S. Cl. 604/509; 128/898; 514/618

(57) ABSTRACT

Provided herein is a method of postconditioning reperfusion of an organ or tissue injured by ischemia in combination with the administration of one or more tissue protective agents that enhance the effect of postconditioning. Also provided is a method of treating a myocardial infarction in a subject to prevent injury to the heart following reperfusion of the heart in combination with the administration of one or more tissue protective agents that enhance the effect of postconditioning.

This application claims priority to U.S. provisional application No. 60/638,461 filed on Dec. 22, 2004. The aforementioned application is herein incorporated by this reference in its entirety.

### BACKGROUND OF THE INVENTION

1. Field of the Invention

The present invention relates to the treatment of organs and tissues injured by ischemia. Specifically, the present invention relates to preventing reperfusion injury in organs and tissues that have suffered an ischemic event.

2. Background Art

Heart disease is the leading cause of premature, permanent disability among American workers, accounting for nearly 20 percent of Social Security disability payments. About 20 million Americans live with the effects of heart disease, and over six million people have heart attacks each year. Every year nearly 50% of patients suffering first-time heart attacks die from myocardial infarctions.

The heart needs a constant and uninterrupted blood supply for normal and continued function. When a patient has a heart attack, the blood flow to part of the heart is stopped, resulting in ischemia. The heart will lose its functional capabilities, and the ischemic part of the heart is in jeopardy of dying, resulting in focal necrosis of the heart tissue. A heart attack can be treated either by percutaneous transluminal coronary angioplasty (PTCA) or by a more invasive procedure, coronary artery bypass graft surgery (CABG). Both procedures can open up a blocked blood vessel (coronary artery) to restore blood supply to the heart muscle, a process called reperfusion. Although the beneficial effects of early reperfusion of ischemic myocardium with thrombolytic therapy, PTCA, or CABG are now well established, an increasing number of studies indicate that reperfusion also induces an additional injury to ischemic heart muscle, such as the extension of myocardial necrosis, i.e., extended infarct size and impaired contractile function and metabolism. Reperfusion injury can extend not only acutely, but also over several days following the heart attack.

Postconditioning is a method of treatment for significantly reducing reperfusion injury to an organ or tissue already undergoing total or subtotal ischemia, wherein the perfusion (blood flow) conditions are modified during the onset of reperfusion. Postconditioning is characterized by a series of brief, iterative interruptions in coronary artery arterial reperfusion applied at the immediate onset of reperfusion. The bursts of reflow and subsequent occlusive interruptions last for a matter of seconds, ranging from 30 second intervals in larger animal models to 10 second intervals in smaller rodent models [50, 51]. Preliminary studies in humans used 1 minute intervals of reperfusion and subsequent interruptions in blood flow during catheter-based percutaneous coronary intervention (PCI) [52].

What is needed in the art is a method of enhancing the beneficial effects of postconditioning to further reduce reperfusion injury in an organ or tissue undergoing total or subtotal ischemia. Therefore, provided herein is a method of enhancing the beneficial effects of postconditioning, comprising administering an effective amount of one or more tissue-protective agents in combination with postconditioning.

### SUMMARY OF THE INVENTION

Provided herein is a method of preventing injury to an organ or tissue in a subject before, during or after reperfusion following an ischemic event to the organ or tissue, comprising a) stopping perfusion of the organ for from about 5 seconds to about 5 minutes; b) resuming perfusion of the organ for from about 5 seconds to about 5 minutes; c) repeating steps a) and b) sequentially for from about 2 to about 50 times; d) allowing uninterrupted perfusion of the organ or tissue; and e) administering to the subject an effective amount of one or more tissue protective agents in a pharmaceutically acceptable carrier, thereby preventing injury to the organ or tissue in the subject.

Also provided is a method of preventing injury to a heart in a subject diagnosed with an ischemic event of the heart, comprising a) clearing a lumen of a coronary artery; b) perfusing the heart for from about 5 seconds to about 5 minutes; c) stopping perfusion of the heart for from about 5 seconds to about 5 minutes; d) repeating steps b) and c) sequentially for from about 2 to about 50 times; e) allowing uninterrupted perfusion of the heart; and f) administering to the subject an effective amount of one or more tissue protective agents in a pharmaceutically acceptable carrier, thereby preventing injury to the heart in the subject.

Provided herein is a method of preventing injury to an organ or tissue in a subject before, during or after reperfusion following an ischemic event to the organ or tissue, comprising a) reducing perfusion of the organ for from about 5 seconds to about 5 minutes; b) resuming perfusion of the organ for from about 5 seconds to about 5 minutes; c) repeating steps a) and b) sequentially for from about 2 to about 50 times; d) allowing uninterrupted perfusion of the organ or tissue; and e) administering to the subject an effective amount of one or more tissue protective agents in a pharmaceutically acceptable carrier, thereby preventing injury to the organ or tissue in the subject.

Also provided is a method of preventing injury to a heart in a subject diagnosed with an ischemic event of the heart, comprising a) clearing a lumen of a coronary artery; b) perfusing the heart for from about 5 seconds to about 5 minutes; c) reducing perfusion of the heart for from about 5 seconds to about 5 minutes; d) repeating steps b) and c) sequentially for from about 2 to about 50 times; e) allowing uninterrupted perfusion of the heart; and f) administering to the subject an effective amount of one or more tissue protective agents in a pharmaceutically acceptable carrier, thereby preventing injury to the heart in the subject.

### BRIEF DESCRIPTION OF THE DRAWINGS

FIG. 1 shows the experimental protocol used to determine the effect of one possible variation in postconditioning on myocardium after ischemia (I) and reperfusion (R). Control group (n=10); Post-con (n=10); Pre-con (n=9): Ischemic preconditioning was elicited by 5 minutes of coronary occlusion followed by 10 minutes of reperfusion before 60 minutes of left anterior descending coronary artery (LAD) occlusion, and postconditioning 3 cycles of 30 seconds of reperfusion followed by 30 seconds of occlusion before 3 hours of reperfusion, respectively. Post-con is postconditioning; pre-con is pre-conditioning.

FIG. 2 is a bar graph showing a reduction in myocardial infarction size by ischemic postconditioning as determined by triphenyltetrazolium chloride (TTC) vs. pre-conditioning staining. Area at risk (AAR) relative to left ventricular (LV) mass (AAR/LV) and area of necrosis (AN) expressed as a percentage of AAR (AN/AAR). Ischemic postconditioning significantly reduced AN/AAR by 48% compared with Control group, and therefore demonstrated equipotent cardioprotection to that of ischemic preconditioning, *P<0.05 vs. Control group. Values are group mean ±S.E.M.

FIG. 3 is a bar graph showing a reduction in myocardial edema in the LAD-perfused myocardium by ischemic postconditioning. Normal: non-ischemic zone; Isch-epi: ischemic subepicardium; Isch-endo: ischemic subendocardium. Ischemic postconditioning significantly reduced tissue water content compared with Control group. *P<0.05 vs. normal zone. \P<0.01 vs. Control group. Values are group mean ±S.E.M.

FIG. 4 is a graph showing the plasma creatine kinase (CK) activity during the course of coronary occlusion and reperfusion. Plasma CK activity was comparable between the two groups at baseline and after ischemia. Consistent with reduction in infarction size, ischemic postconditioning significantly decreased CK activity starting at 2 hours of reperfusion relative to the Control group values. Values are mean ±S.E.M.; *P<0.01 vs. Baseline and Isch values. p<0.05 vs. Control group.

FIG. 5 is a line graph showing regional transmural myocardial blood flow in the ischemic-reperfused myocardium. Values at baseline and during ischemia were comparable between the two groups. Hyperemia at 15 minutes of reperfusion was significantly inhibited by ischemic pre- and postconditioning. Values are mean ±S.E.M. *P<0.05 vs. ischemia=\P<0.05 vs. Control group.

FIG. 6 is a line graph showing post-ischemic-reperfusion endothelium function of non-ischemic left circumflex coronary artery (LCX) coronary artery rings and ischemic-reperfused (LAD) coronary artery rings assessed as responses to incremental concentrations of acetylcholine in organ chambers. Responses to acetylcholine at reperfusion were significantly blunted vs. responses of the non-ischemic LCX coronary artery rings. Response in ischemic postconditioning was significantly increased, suggesting better endothelial function and avoidance of ischemic-reperfusion injury with postconditioning. Values are Mean ±S.E.M. of at least 12 rings from 5 dogs. *P<0.05 LAD in Control group vs. ischemic post- and pre-conditioning.

FIG. 7 is a line graph showing responses of non-ischemic LCX coronary rings and ischemic-reperfused (LAD) coronary rings to the vascular smooth muscle vasodilator, nitroprusside. No group difference was detected in all groups, suggesting that vascular smooth muscle function was normal and comparable among groups.

FIG. 8 is a bar graph showing the inhibition in adherence of unstimulated fluorescence-labeled neutrophils to coronary endothelium by ischemic postconditioning vs. pre-conditioning. The degree of adherence correlates with the degree of damage sustained by the coronary artery endothelium, related to loss of basal generation of nitric oxide or adenosine. LCX: non-ischemic left circumflex coronary artery; LAD: ischemic/reperfused left anterior descending coronary artery; Post-LAD: LAD in ischemic postconditioning group; Pre-LAD: LAD in ischemic pre-conditioning group. As potent as the protection by ischemic preconditioning, ischemic postconditioning significantly inhibited neutrophil adherence to coronary endothelium compared with Control group. Values are group mean ±S.E.M. *P<0.05 vs. LCX; H P<0.01 vs. LAD in Control group.

FIG. 9 shows tissue myeloperoxidase (MPO in delta absorbance A units/minute, (abs/min.)) activity as a marker of neutrophil accumulation in non-ischemic (Normal) and ischemic zones in the different experimental groups after LAD ischemia and reperfusion. Increased MPO activity was seen at the end of reperfusion in the control AAR. Ischemic postconditioning significantly decreased MPO activity compared with Control group, and was comparable to that in the preconditioning group. Bar height represents mean ±SEM. *p<0.05 vs. normal tissue; \p<0.05 Post-con and Pre-con group vs. Control group.

FIG. 10 shows a schematic diagram of the study protocol in a rat model of ischemia-reperfusion. Cross-hatched bar=time when the sodium-hydrogen exchange inhibitor (NHE-1), cariporide is administered intravenously. Vertical hatched bar=postconditioning algorithm. Control (n=8); occlusion of the left coronary artery (LCA) for 30 min (dark bar), was followed by 3 h of reperfusion (open bar). Post-con (n=8); 10 of full reperfusion (R, open bar) and 10 s of re-occlusion ischemia (I, dark bar) were repeated for three cycles. NHE(1) (n=8); cariporide (1 mg/kg) was injected 5 min prior to reperfusion, followed by unbridled R. NHE(1)+Post-con (n=8); cariporide followed by Post-con. Delay (D)-NHE(1) (n=8); cariporide was injected for 5 min after 1 min of full unbridled reperfusion. Post-con+D-NHE(1) (n=8); Post-con followed by injecting cariporide for 5 min. Post-con=postconditioning, NHE(1)=1 mg/kg cariporide

FIG. 11 shows the area at risk (AAR) expressed as a percentage of the left ventricle (LV) and the area of necrosis (AN) expressed as a percentage of the AAR. Infarct size is expressed as a percentage of AN and AAR. In all groups, infarct size decreased compared to that of Control. The decrease in infarct size observed in Post-con+D-NHE(1) group was significantly greater than postconditioning alone. *p<0.05 vs. Control, #p<0.001 vs Control. Post-con=postconditioning. Values are means ±SEM.

### DETAILED DESCRIPTION OF THE INVENTION

It must be noted that, as used in the specification and the appended claims, the singular forms “a,” “an,” and “the” include plural referents unless the context clearly dictates otherwise. Thus, for example, reference to “an agent” includes multiple copies of the agent and can also include more than one particular species of agent.

Provided herein is a method of minimizing damage in an ischemic/reperfused heart muscle by providing a protective effect when it is applied in the treatment of ischemic heart disease in conjunction with percutaneous transluminal coronary angioplasty (PTCA) and/or coronary artery bypass grafting surgery (CABG). The method (postconditioning), in combination with the administration of one or more tissue protective agents, can be applied in other clinical situations, for example, following organ transplantation when the donor organ has suffered temporary ischemia, renal angioplasty, and ablation of cerebral or peri-cerebral thromboses. Moreover, postconditioning can be applied in conjunction with pharmacological therapy, or mimicked by pharmacological therapy utilizing mediators of the mechanisms involved in postconditioning. As used herein, “postconditioning reperfusion” means the application of repeated cycles of stopping or reducing perfusion followed by resuming perfusion of an organ or tissue previously affected by ischemia. As used herein, “perfusion” and “perfusing” mean blood flow to, through or within an organ or tissue. As used herein, “reperfusion” is the restoration or resumption of blood flow to, through or within an organ or tissue after a period of interruption of blood flow to, through or within the organ or tissue.

As used herein, “injury” means damage or potential damage or dysfunction of an organ or tissue as evidenced by, for example, edema (swelling), loss of function and/or infiltration of the organ or tissue by leukocytes, necrosis and/or apoptosis. An injury can be as minimal, for example, as barely perceptible swelling of the cells comprising the organ or tissue. Further, an injury can include damage to an organ or tissue that occurs during and/or after a period of ischemia (an ischemic event) or after a period of reperfusion (reperfusion injury). As used herein, an “injured” or “target” organ or tissue is an organ or tissue that has had or may have some potential damage from ischemia or reperfusion. A “leukocyte” can be a neutrophil, lymphocyte, monocyte, macrophage, basophil or eosinophil. As used herein, “ischemia” means an interrupted supply of blood to an organ or tissue that can be caused by, for example, a mechanical obstruction (i.e., a thrombus or embolus) in an artery, external compression of an artery, constriction of an artery caused by vasospasm, iatrogenic blocking of blood flow in an artery to an organ (e.g., an organ that is to be surgically removed from one subject and subsequently transplanted into another subject), and/or hypotension (low blood pressure). Hypotension can result from a cardiac arrhythmia, a neurogenic reflex causing vasodilation and subsequent pooling of blood in the lower extremities (e.g., a vasovagal reflex), hypovolemia (i.e., a reduced amount of intravascular fluid) caused by inadequate fluid intake by a subject or loss of blood by a subject following a traumatic wound. Thus, an “ischemic injury” means the damage or potential damage to an organ or tissue that results from the interruption of blood flow to the organ or tissue, i.e., an ischemic event. As used herein, a “reperfusion injury” is the damage or potential damage to an organ or tissue that results from the resumption of blood flow to the organ or tissue during or following an ischemic event. An “ischemic event” is an interruption of the blood supply to an organ or tissue. As used herein, a “total” ischemic event is a complete interruption of the blood supply to an organ or tissue. As used herein, a “subtotal” ischemic event is an incomplete interruption of the blood supply to an organ or tissue. Examples of an organ or tissue that can be subject to an ischemic event and/or suffer an ischemic injury include, but are not limited to, heart, brain, eye, kidney, intestine, pancreas, liver, lung and skeletal muscle.

Further, in the case of organ or tissue transplantation, wherein a subject receives an organ or tissue from a donor, the disclosed methods can be used after the transplanted organ is implanted into the recipient and the vascular attachments have been completed. Examples of organs that can be treated with postconditioning include, but are not limited to, lung, liver, pancreas, heart and kidney.

Thus, provided is a method of preventing injury to an organ or tissue in a subject during or after reperfusion following an ischemic event to the organ or tissue, comprising: a) stopping perfusion of the organ or tissue for from about 5 seconds to about 5 minutes; b) resuming perfusion of the organ or tissue for from about 5 seconds to about 5 minutes; c) repeating steps a) and b) sequentially for from about 2 to about 50 times; and d) ending stopping perfusion of the organ or tissue, thereby preventing injury to the organ or tissue in the subject during or after reperfusion following an ischemic event.

Also provided herein is a method of preventing injury to an organ or tissue in a subject before, during or after reperfusion following an ischemic event to the organ or tissue, comprising: a) reducing perfusion of the organ or tissue for from about 5 seconds to about 5 minutes; b) resuming perfusion of the organ or tissue for from about 5 seconds to about 5 minutes; c) repeating steps a) and b) sequentially for from about 2 to about 50 times; and d) ending reducing perfusion of the organ or tissue, thereby preventing injury to the organ or tissue in the subject during or after reperfusion following an ischemic event. As used herein, “reducing perfusion” means reducing the amount of perfusion with blood or other fluids such that injury to the organ or tissue is prevented. For example, reducing perfusion to about 20%, 15%, 10% or 5% of the expected blood flow is contemplated. Also contemplated is a combination of stopping and reducing perfusion in a single procedure.

As used herein, a subject can include domesticated animals, such as cats, dogs, etc., livestock (e.g., cattle, horses, pigs, sheep, goats, etc.), laboratory animals (e.g., mouse, rabbit, rat, guinea pig, etc.) and birds. Preferably, the subject is a mammal such as a primate, and, more preferably, is a human.

As provided herein, after reperfusion has been established, injury to an organ or tissue undergoing ischemia can be prevented by repeatedly stopping or reducing perfusion of the organ or tissue and then resuming perfusion of the organ or tissue. A cycle of stopping or reducing perfusion and resuming perfusion can be repeated for from about two to about 50 times. Stopping or reducing perfusion of the organ or tissue can last for from about 5 seconds to about 5 minutes, followed by resumption of perfusion of the organ or tissue that lasts for from about 5 seconds to about 5 minutes. The duration of the stoppages of blood flow can either increase or decrease during the procedures, i.e. the first cycle of reperfusion can last 30 seconds and the stoppage 30 seconds, but successive cycles can last 20 seconds of reperfusion followed by 40 seconds of stoppage, the succeeding cycle 10 seconds of reperfusion followed by 50 seconds of ischemia. Alternatively the duration of stoppage can decrease as the cycles progress. After the last cycle of stopping or reducing and starting perfusion, blood flow to the organ or tissue is restored unabated, or can be under some degree of control. For example, after the last reperfusion-stoppage cycle, blood flow can be started slowly and gradually increased until normal blood flow is achieved. A person of skill can use algorithms known in the art to determine the rate at which blood flow can be resumed.

A person of skill can stop or reduce perfusion of an organ or tissue by introducing into the lumen of a blood vessel that supplies blood to the organ or tissue a mechanical device that can be used to temporarily block blood flow in the vessel. After a selected period of time, the device can be manipulated to restore perfusion of the organ or tissue. After performing a selected number of cycles of stopping or reducing perfusion and resuming perfusion of the organ or tissue, a person of skill can remove the device from the lumen of the blood vessel so that reperfusion (i.e., blood flow to the organ or tissue) is restored. The blood vessel can be an artery or a vein, preferably an artery.

An example of a mechanical device that can be used in postconditioning reperfusion is a catheter to which is attached a medical balloon that can be inflated within the lumen of a vessel to block blood flow to the injured organ or tissue and deflated to restore blood flow to the injured organ or tissue. A catheter/balloon device can be introduced into a blood vessel of a subject either percutaneously or directly into a vessel during an operative procedure. After the catheter/balloon is within a vessel lumen, a person of skill can guide it to a specific artery under radiologic control according to well known methods.

In another aspect, a hollow catheter can be introduced into a vessel of a subject. The diameter of the lumen of the catheter can be large enough to permit blood, fluid or a blood/fluid combination to flow through it to the targeted organ or tissue. The catheter can be attached to a pump that is external to the subject. The pump can be activated to pump blood, crystalloid fluids or a combination of blood in crystalloid fluids through the catheter to the targeted organ or tissue and inactivated to stop or reduce blood flow to the targeted organ or tissue. After reperfusion of an organ or tissue that has suffered an ischemic injury has been established, a person of skill can inactivate the pump to stop or reduce perfusion of the targeted organ or tissue. After a selected period of time, for example, from about 5 seconds to about 5 minutes, a person of skill can activate the pump to begin perfusion of the targeted organ for from about 5 seconds to about 5 minutes. The pump can be used to stop or reduce, and start perfusion of the targeted organ or tissue for from about two to about 50 cycles. After postconditioning by catheter perfusion techniques has been completed, the catheter can be removed from the subject. This can also be applied during on-pump surgery in which the pump can be used to deliver cardioplegia or other surgical solutions, or during transplantation of any organ, i.e. liver, lung, pancreas, or kidney.

In another aspect, after reperfusion has been established, a medical practitioner can stop or reduce blood flow to an organ or tissue injured by ischemia, using external compression of the vessel. The practitioner can use a gloved hand, a ligature, an external pump, or a surgical instrument, for example, a clamp or hemostat, to temporarily stop or reduce blood flow through the vessel to the injured organ or tissue. After blood flow through the vessel has been stopped or reduced for a selected period of time, the practitioner can remove the hand, the ligature, the external pump, or the surgical instrument from the vessel, thereby removing the interruption of blood flow to the injured organ or tissue. After a selected number of cycles of temporarily stopping or reducing, and restoring perfusion of the injured organ or tissue, the practitioner can restore blood flow to the organ or tissue without further intervention. An example of this application of the treatment is off-pump cardiac surgery in which the surgeon loosens and subsequently tightens the ligature on the target vessel undergoing bypass as a form of postconditioning. This can also be applied during on-pump surgery, or during transplantation of any organ, i.e. liver, lung, pancreas, or kidney.

Before, during or after postconditioning reperfusion of an organ or tissue previously affected by ischemia, a practitioner can administer to the subject an effective amount of a tissue protective agent in a pharmaceutically acceptable carrier that can further prevent injury to the organ or tissue. Thus, provided herein is a method of preventing injury to an organ or tissue in a subject before, during or after reperfusion following an ischemic event to the organ or tissue, comprising a) stopping perfusion of the organ for from about 5 seconds to about 5 minutes; b) resuming perfusion of the organ for from about 5 seconds to about 5 minutes; c) repeating steps a) and b) sequentially for from about 2 to about 50 times; d) allowing uninterrupted perfusion of the organ or tissue; and e) administering to the subject an effective amount of one or more tissue protective agents in a pharmaceutically acceptable carrier, thereby preventing injury to the organ or tissue in the subject.

Also provided is a method of preventing injury to an organ or tissue in a subject during or after reperfusion following an ischemic event to the organ or tissue, comprising a) reducing perfusion of the organ for from about 5 seconds to about 5 minutes; b) resuming perfusion of the organ for from about 5 seconds to about 5 minutes; c) repeating steps a) and b) sequentially for from about 2 to about 50 times; d) allowing uninterrupted perfusion of the organ or tissue; and e) administering to the subject an effective amount of one or more tissue protective agents in a pharmaceutically acceptable carrier, thereby preventing injury to the organ or tissue in the subject.

Also provided herein is a method of preventing injury to a heart in a subject diagnosed with an ischemic event of the heart, comprising a) clearing a lumen of a coronary artery; b) perfusing the heart for from about 5 seconds to about 5 minutes; c) stopping perfusion of the heart for from about 5 seconds to about 5 minutes; d) repeating steps b) and c) sequentially for from about 2 to about 50 times; e) allowing uninterrupted perfusion of the heart; and f) administering to the subject an effective amount of one or more tissue protective agents in a pharmaceutically acceptable carrier, thereby preventing injury to the heart in the subject.

Further provided herein is a method of preventing injury to a heart in a subject diagnosed with an ischemic event of the heart, comprising a) clearing a lumen of a coronary artery; b) perfusing the heart for from about 5 seconds to about 5 minutes; c) reducing perfusion of the heart for from about 5 seconds to about 5 minutes; d) repeating steps b) and c) sequentially for from about 2 to about 50 times; e) allowing uninterrupted perfusion of the heart; and f) administering to the subject an effective amount of one or more tissue protective agents in a pharmaceutically acceptable carrier, thereby preventing injury to the heart in the subject.

As used herein, an “ischemic-reperfusion event” of a heart (heart attack) is an event that occurs when the heart muscle (myocardium) suffers an interruption in its blood supply (ischemia) that is ultimately followed by restoration of blood flow (reperfusion). During ischemia, the muscle rapidly loses function, is depleted of its energy supply and undergoes changes consistent with inflammation. A second, more robust or explosive injury occurs at the onset of reperfusion (i.e., reperfusion injury), characterized by an increase in inflammation, activation of white blood cells in the region of the heart, tissue edema and swelling, injury to the small blood vessels feeding the heart muscle in the area involved in the heart attack, an extension of necrosis (cell death) to include greater amounts of heart tissue, and apoptosis. By “myocardial infarction” is meant an ischemic-reperfusion injury to the heart in which part of the myocardium has undergone necrosis or apoptosis, i.e., programmed cell death. Therefore, injury to the heart during a heart attack occurs during both ischemia and reperfusion.

An evolving heart attack reflects the dynamic nature of injury during both ischemia and reperfusion. Thus, the injury that started or was triggered by ischemia can continue after the onset of reperfusion in which cell function can further deteriorate, and the amount of muscle actually going on to die increases with reperfusion. There is a clear relationship between ischemic injury and reperfusion injury in that the ischemic event sets the stage for reperfusion injury. The more severe the ischemic event is, the more severe the subsequent reperfusion injury is. Hence, the two events are often referred to as ischemia-reperfusion injury to reflect this intimate link between two separate but interrelated events. Interventions can be directed to either a decrease in ischemic injury or a decrease in reperfusion injury.

It is contemplated that a subject who presents to a medical facility with signs and symptoms of a heart attack can be diagnosed in time to be treated according to the methods taught herein. If during angiographic examination of the subject's coronary arteries it is determined that a coronary artery is blocked (partially or totally) by a thrombus, embolus, cholesterol plaque or other obstruction and that the blocked artery can be opened by percutaneous transluminal coronary angioplasty (PTCA), the practitioner can insert a balloon catheter percutaneously into a femoral vein of the subject and guide the catheter into the blocked coronary artery. After the balloon is properly localized at or near the site of blockage of blood flow in the coronary artery, the practitioner can manipulate and/or inflate the balloon to compress the thrombus, embolus, cholesterol plaque or other obstruction against the vessel wall, thereby clearing the lumen and reperfusing the myocardium.

To prevent injury and/or subsequent injury to the injured myocardium after reperfusion has been established, postconditioning can be performed. Specifically, the practitioner can leave the balloon catheter in place and re-inflate the balloon for from about 5 seconds to about 5 minutes to stop or reduce perfusion of the injured myocardium. After the selected time period of stopped or reduced perfusion, the practitioner can deflate the balloon to restore perfusion of the myocardium for from about 5 seconds to about 5 minutes. This cycle of inflating and deflating the balloon within the lumen of the coronary artery can, for example, be repeated for from about 2 to about 50 times. After the final deflation of the balloon, the practitioner removes the balloon catheter.

In another aspect, a subject diagnosed with an ischemic event and found to have coronary artery disease not amenable to PTCA can be treated with CABG surgery. During the operative procedure and after the diseased coronary artery has been bypassed to restore blood flow to the myocardium, a surgeon can effect postconditioning reperfusion by stopping or reducing perfusion of the injured myocardium by compressing the grafted vessel with a gloved hand, a ligature, an external pump, or with a surgical instrument, for example, a clamp or a hemostat. Stopping or reducing perfusion can be maintained for from about 5 seconds to about 5 minutes. After the selected period of time has passed, the surgeon can remove the hand, the ligature, the external pump, or the surgical instrument from the vessel, thereby restoring blood flow through the graft to the injured myocardium. Perfusing the injured myocardium can last for from about 5 seconds to about 5 minutes. The cycle of stopping or reducing perfusion and resuming perfusion of the injured myocardium can be repeated for from about two to about 50 times. At the end of the last cycle, perfusion of the injured myocardium is maintained.

A person of skill can enhance the effects of PTCA and CABG by administering a compound comprising an effective amount of one or more tissue protective agents in combination with postconditioning. The compound can be administered prior to, during or immediately after postconditioning. Optionally, the tissue protective agent can be administered immediately before a blocked lumen is cleared. As used herein, “optionally” means that the subsequently described event or circumstance may or may not occur, and that the description includes instances where said event or circumstance occurs and instances where it does not.

As used herein, an “effective amount” of a tissue protective agent of this invention is that amount needed to achieve the desired result or results known to those skilled in the art. An example of an organ or tissue that can have the desired results of postconditioning reperfusion is the heart, in which reduction in infarct size, decrease in myocardial edema, attenuation in release of creatine kinase, inhibition of hyperemia during early reperfusion, augmentation in endothelium-dependent vascular relaxation, decrease in neutrophil adherence to ischemic/reperfused coronary endothelium, increased contractile function and decrease in neutrophil accumulation in ischemic myocardium can be monitored and attained. Thus, a heart treated according to the disclosed methods can exhibit better overall function, for example, increased cardiac output and smaller heart size due to less severe heart failure, fewer arrhythmias and a steadier heart rate. Moreover, a subject can exhibit better tolerance to exercise and can better tolerate a subsequent heart attack.

By a “pharmaceutically acceptable carrier” is meant a material that is not biologically or otherwise undesirable, i.e., the material can be administered to an individual along with the protective agent without causing substantial deleterious biological effects or interacting in a deleterious manner with any of the other components of the composition in which it is contained. Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution can be from about 5 to about 8, and can be, for example, from about 7 to about 7.5. Further carriers include sustained release preparations such as semipermeable matrices of solid hydrophobic polymers containing the agent, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intravenously, intra-arterially, intramuscularly, subcutaneously or intraperitoneally. Other compounds will be administered according to standard procedures used by those skilled in the art.

Pharmaceutical compositions can include carriers, thickeners, diluents, buffers, preservatives, surface-active agents and the like in addition to the molecule of choice. Pharmaceutical compositions can also include one or more active ingredients such as antimicrobial agents, anti-inflammatory agents, anesthetics, and the like.

The pharmaceutical composition containing the tissue protective agent can be administered in a number of ways depending on whether local or systemic treatment is desired, and on the area to be treated. Administration can be orally, by inhalation, or parenterally, for example by intravenous drip, intra-arterial, subcutaneous, intraperitoneal, intramuscular injection, or intravascular injection/infusion. Compositions for oral administration include powders or granules, suspensions or solutions in water or non-aqueous media, capsules, sachets, or tablets. Thickeners, flavorings, diluents, emulsifiers, dispersing aids or binders can be desirable.

In one aspect, a tissue protective agent can be administered through a catheter within the lumen of a vessel (intravascular injection/infusion) near the site where the vessel enters the injured organ or tissue, or can be administered parenterally, i.e., intravenously or in an artery.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Examples of tissue protective agents that can be used with the disclosed methods include, but are not limited to, phosphodiesterase-5 inhibitors, agents that increase cAMP or cGMP, opioids, PKC stimulators (specifically PKC epsilon (ε)), PAR2 agonists, sodium/hydrogen exchange (NHE-1) inhibitors; anti-inflammatory agents; anti-oxidants, protease inhibitors; sodium channel blockers; K_{ATP} channel regulating agents; calcium channel antagonists and regulators; regulators of thrombosis; metabolic enhancing agents; buffering agents and regulators; endothelin-1 antagonists, inhibitors and regulators; inhibitors of apoptosis; mitochondrial permeability transition pore opening inhibitors; signal transduction stimulators and inhibitors; anesthetics; and statins.

The dosage and route of administration of a tissue protective agent will depend on the specific agent used. A list of exemplary tissue protective agents and their respective dosages that can be administered in combination with postconditioning is disclosed below (Table 1). A person of skill can administer a compound comprising one or more tissue protective agents to a subject in need of treatment according to the methods disclosed. A person of ordinary skill in the art would know the appropriate dosage and route of administration of a tissue protective agent and can vary the dosage according to the age, weight, mode of injection/infusion (intramuscular, intravascular, local, systemic), gender and overall condition of the subject, using only routine experimentation given the teachings herein (see, e.g., Remington's Pharmaceutical Sciences, Martin, E. W. (ed.), latest edition. Mack Publishing Co., Easton, Pa.). For example, the dosage of intravenous heparin, an anticoagulant, can be from about 10 units to about 10,000 units.

A further example of a tissue protective agent that can be used with the disclosed methods is a sodium/hydrogen exchange (NHE-1) inhibitor, in a pharmaceutically acceptable carrier. An example of an NHE-1 inhibitor is cariporide which can be administered in an intravascular solution in a concentration of 0.1-15 μM, or 3 mg/Kg by intravenous bolus, or 120 mg three times daily, or 1 mg/Kg-10 mg/Kg. Another example of an NHE-1 inhibitor is eniporide which can be administered in an intravascular solution in a concentration of 0.5-15 μM. Eniporide can be administered at 3 mg/kg either before coronary artery occlusion (ischemia) or just prior to or concomitant with onset of reperfusion. The eniporide can be given as a one-time bolus or continued for one to three hours as an infusion of 3 mg/kg/hour. Eniporide can be given as a 1-200 mg intravenous infusion over a ten-minute period.

The following examples are put forth to provide those of ordinary skill in the art with a complete disclosure and description of how the compositions and/or methods claimed herein are made and evaluated, and are intended to be purely exemplary of the invention and are not intended to limit the scope of what the inventors regard as the invention. Efforts have been made to ensure accuracy with respect to numbers (e.g., amounts, temperature, etc.), but some errors and deviations should be accounted for. The present invention is more particularly described in the following examples which are intended as illustrative only because numerous modifications and variations therein will be apparent to those skilled in the art.

### EXAMPLE 1

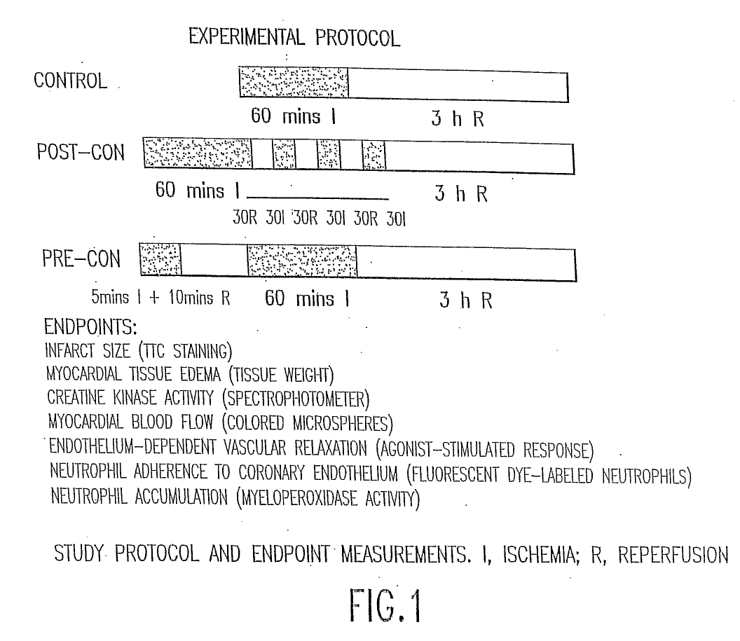

The concept of postconditioning was tested in an opened-chest canine model of regional myocardial ischemia and reperfusion. All animals were randomly assigned to one of the following three groups (FIG. 1): 1) Control: the left anterior descending coronary artery (LAD) was reversibly occluded for 60 minutes, and the ischemic myocardium was then reperfused for 3 hours; 2) ischemic postconditioning (Post-con): after 60 minutes of LAD occlusion, the ischemic myocardium was initially reperfused using 3 cycles of repetitively applied reperfusion followed by re-occlusion of the coronary artery, i.e., 30 seconds of reperfusion followed by 30 seconds of occlusion repeated in 3 successive cycles; 3) ischemic preconditioning (Pre-con): 5 minutes of LAD occlusion and 10 minutes of reperfusion were performed before the 60 minutes of myocardial ischemia.

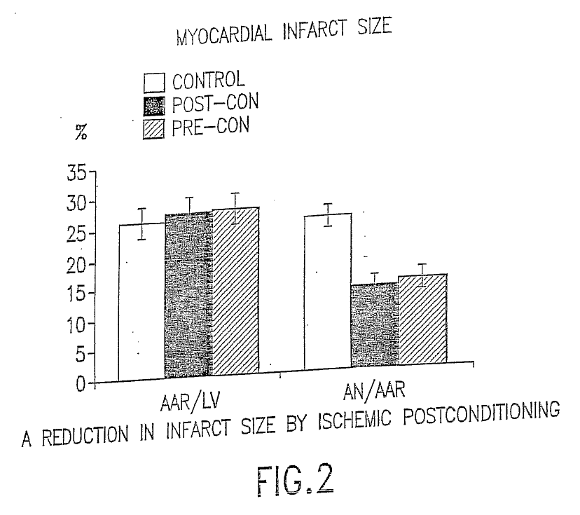

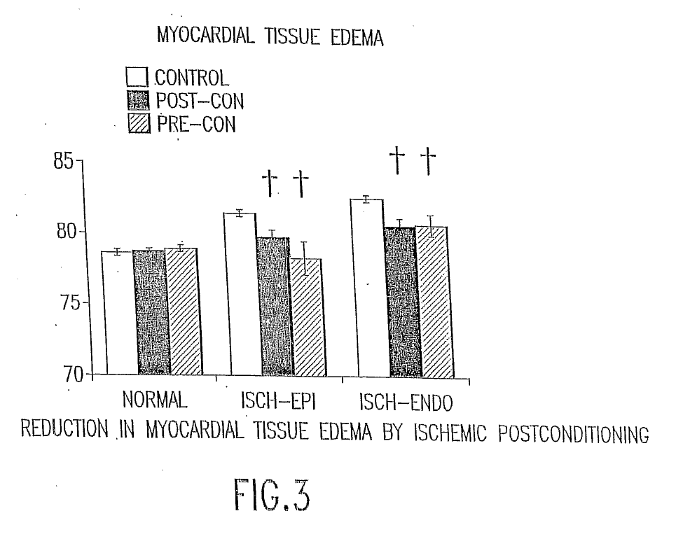

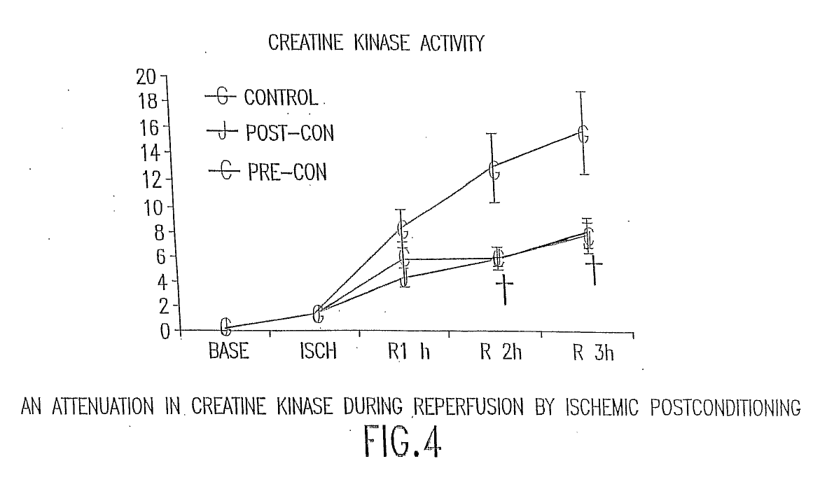

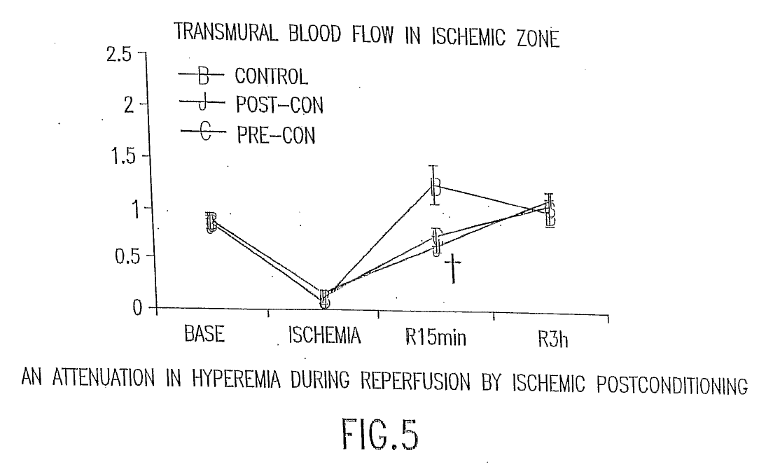

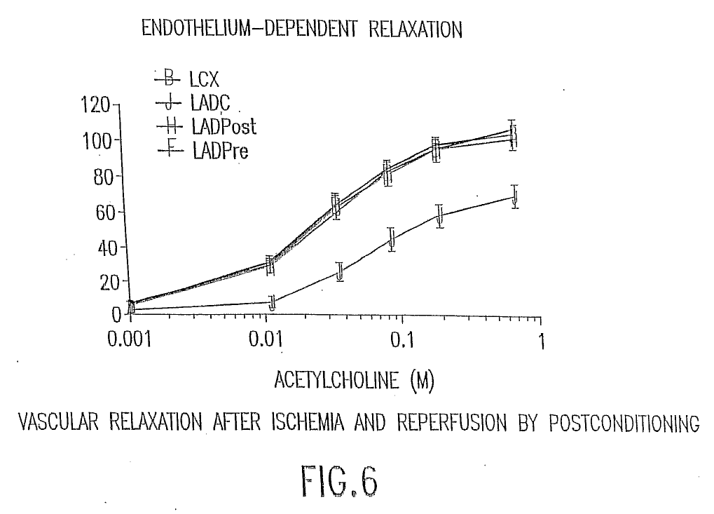

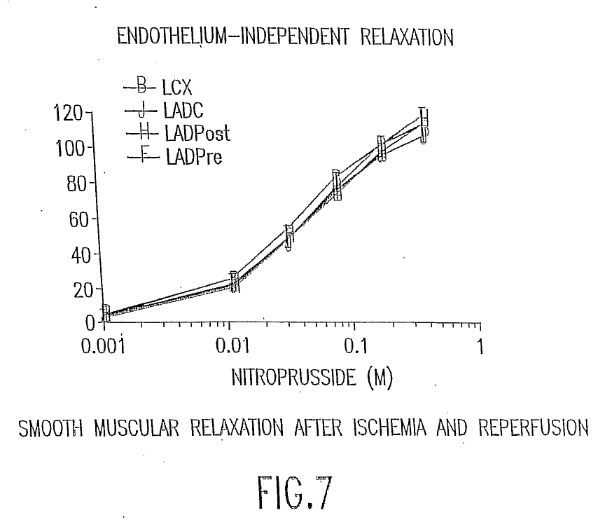

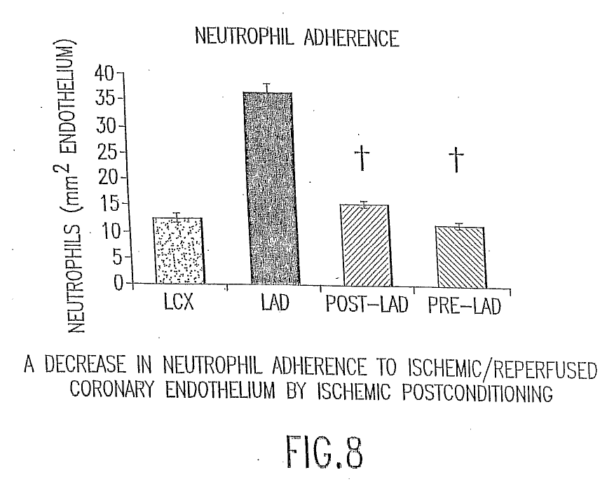

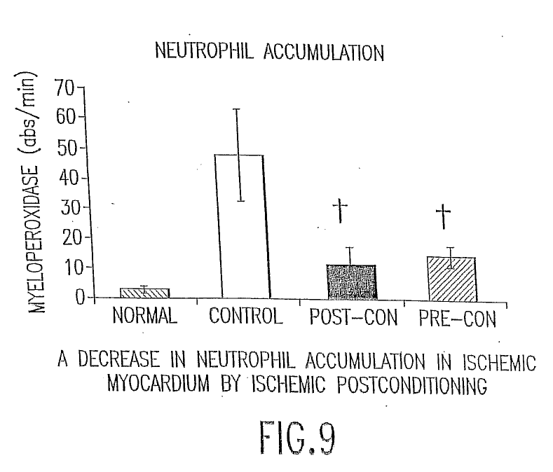

FIGS. 1-9 show the salutary effects of postconditioning on the ischemic/reperfused heart. Those effects include reduction in infarct size measured by a vital stain (triphenyltetrazolium chloride) post-mortem [6], which was confirmed by a decrease in the release of creatine kinase measured spectrophotometrically from arterial blood plasma [6]. Creatine kinase is an intracellular macromolecule which escapes from a cell only when there is severe, lethal injury to that cell.

Moreover, postconditioning is associated with a decrease in myocardial edema in the previously ischemic myocardium, as measured by tissue dessication. Tissue edema (water gain) occurs when the microvasculature is severely injured and fails to retain blood fluids in the vascular space. Fluid that has leaked into the myocardium can surround and compress those injured capillaries, further reducing blood flow to the heart muscle. This vascular injury has been associated with irreversible injury to the myocardium, e.g., necrosis.

Postconditioning also inhibits post-ischemic hyperemia during early reperfusion as measured by an electronic blood flow probe placed around the target coronary artery, suggesting that there is sufficient oxygen delivery during those brief periods of intermittent perfusion to satisfy myocardial energy demands.

Postconditioning is associated with a significantly greater endothelium-dependent vascular relaxation response to acetylcholine, as measured by in vitro techniques. Acetylcholine is an endothelial-specific stimulator of the vasorelaxant agent, nitric oxide [7]. The endothelium of coronary arteries, arterioles and venules is extraordinarily sensitive to reperfusion injury and undergoes obliteration within the first few moments of reperfusion, and the obliteration continues for hours after the onset of reperfusion. Salvage of the vascular endothelium is important because a healthy endothelium prevents abnormalities in blood flow regulation and prevents a localized vascular inflammatory response, thereby preventing triggering migration of neutrophils into the previously ischemic zone and the formation of blood clots in the artery. Blood clots in the reperfused vessels can cause a secondary ischemia and can ultimately lead to death of the heart tissue. The decrease in neutrophil adherence to ischemic/reperfused coronary endothelium, measured by fluorescence microscopy, also represents improvement in post-ischemic endothelial function with postconditioning.

Further, postconditioning attenuated neutrophil accumulation in ischemic myocardium, as measured by the myeloperoxidase (MPO) assay of tissue samples from the post-reperfusion myocardium. This suggests that postconditioning reduced the inflammatory response to ischemia/reperfusion which has been associated with the pathogenesis of infarction, contractile dysfunction and apoptosis.

### EXAMPLE 2

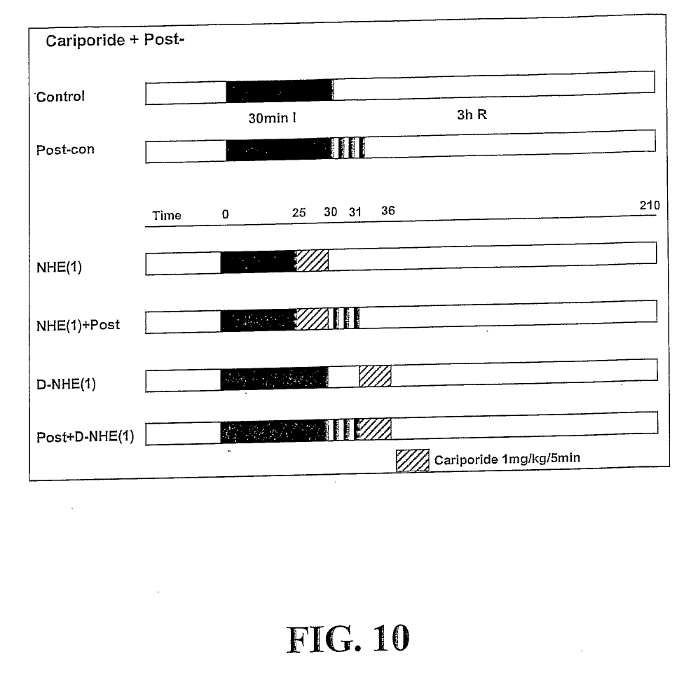

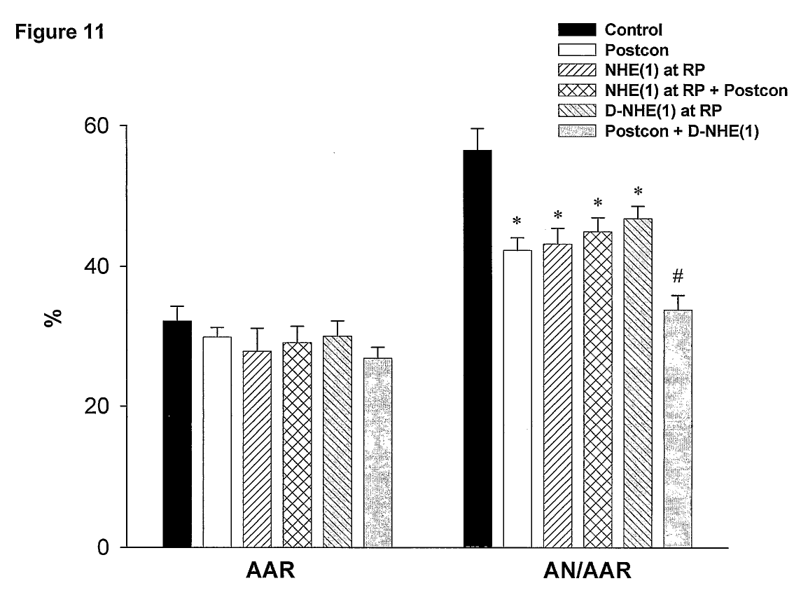

Postconditioning can be enhanced by pharmacological means which capture the protective actions of the proximal mediators such as adenosine and opioids. In a rat model of myocardial infarction, it can be shown that the dual approach of attenuating reperfusion injury by applying postconditioning in the presence of a sodium-hydrogen exchange (NHE-1) inhibitor during early reperfusion achieves greater infarct size reduction than either intervention alone [48]. Specifically, in an anesthetized rat model, the left coronary artery (LCA) was occluded for 30 min of ischemia (I) and reperfused for 3 hours. Rats were randomly divided into six groups (n=8 each) (FIG. 10): Control: no intervention at reperfusion; Postconditioning: three cycles of 10-s reperfusion followed by 10-s re-occlusion were applied during the first minute of reperfusion; NHE(1): cariporide (1 mg/kg) was infused 5 min before reperfusion, with or without postconditioning; Delayed (D)-NHE(1): a 5-minute infusion of cariporide was begun 1 min after onset of reperfusion equivalent to postconditioning period; Post-con+D-NHE(1): Completion of postconditioning was followed immediately by cariporide infusion for 5 min. The infarct size reduction with postconditioning was comparable to that observed with NHE(1) inhibitor alone (42±2 vs. 43±2%, respectively) (FIG. 11). When NHE(1) preceded postconditioning as a reperfusion intervention, infarct size was not further reduced (45±2%*) compared to either intervention alone. However, an additive reduction in infarct size was achieved when postconditioning preceded NHE(1) inhibitor at reperfusion (Post-con+D-N-HE(1), 34±2%). Thus, infarct size reduction by postconditioning is enhanced by NHE inhibition at reperfusion when postconditioning precedes cariporide administration.

### EXAMPLE 3

Adenosine is a mediator of the cardioprotection of postconditioning. Isolated-perfused mouse hearts were subjected to 20 min global ischemia (I) and 30 min reperfusion (R) with or without Postcon (6 cycles of 10 sec. R & occlusion). Intravascular purines in coronary effluent were analyzed by HPLC. To determine whether endogenous adenosine played a physiological role in postconditioning, the left coronary artery (LCA) was occluded for 30 min and reperfused for 3 hours in anesthetized open-chest rats. The rats were randomly divided into six groups (n=8 each): Control (no intervention); postconditioning (3 cycles of 10-s R followed by 10-s LCA I before 3 hours R); 8-SPT (subtype non-selective adenosine receptor antagonist, 10 mg/kg), or ZW241385 (A2a receptor antagonist, 0.2 mg/kg), was given 5 min before R with or without postconditioning.

In mouse hearts, postconditioning decreased effluent [adenosine] at 2 min R (58±5* vs 155±16 nM/min/g), and improved contractile function (LV developed pressure 32±7* vs 16±2 mmHg) and end-diastolic pressure (27±3* vs 36±3 mmHg) at 5 min R which persisted at 30 min R. In in vivo rats, postconditioning reduced infarct size (TTC) vs the control group (40±3.1%* vs 52±2.2%). 8-SPT (51±2.5%) or ZM241385 alone (50±2.1%) without postconditioning had no effect on infarct size. The infarct-sparing effect of postconditioning was abrogated by 8-SPT and ZM241385 (50±1.8% and 49±2.6%). Neutrophil (PMN) accumulation (myeloperoxidase activity) in the area at risk was less in postconditioning vs Control (1.0±0.2* vs 2.2±0.4 U/100 g protein); adenosine receptor antagonists blocked the reduction of PMN accumulation in postconditioning (postconditioning+8-SPT 2.1±0.2; postconditioning+ZM241385 1.6+0.2).

Postconditioning increases retention time and intravascular content of endogenous adenosine during early R, which can reduce infarct size by A2a receptor-mediated mechanisms. *p<0.05 vs Control.

### EXAMPLE 4

In a rabbit model of myocardial infarction it was found that postconditioning ischemia for 20 s, but not 10 s reduced infarct size (20±3% and 34±3% of the left ventricular area at risk, respectively) as compared with control (41±2%). Exposure to 1.0, but not 0.5, minimum alveolar concentration isoflurane decreased infarct size (21±2% and 43±3%, respectively). However, combined postconditioning (10 s) and 0.5 minimum alveolar concentration isoflurane markedly reduced infarct size (17±5%) [49].

Administration of 0.5 MAC isoflurane, a concentration of this agent that does not provide cardioprotection alone, reduces the time threshold of brief ischemic stimuli required to produce postconditioning, or in other words enhances postconditioning protection. Thus, pharmacological postconditioning can be successfully applied during the early moments of reflow.

### EXAMPLE 5

Thirty patients undergoing percutaneous coronary intervention for evolving acute myocardial infarction with angioplasty and deployment of stents were randomized to either a control group which received no further intervention, or to postconditioning in which the angioplasty balloon was deflated for 1 minute and re-inflated for 1 minute, repeated for 4 intervals commencing immediately after reperfusion was restored by the angioplasty-stent procedure. There were no adverse events in the postconditioning group. Postconditioning reduced infarct size estimated by area under the 72-hour creatine kinase curve in hospital, significantly compared to the Control group (208,984±26,576 vs 326,095±48779 arbitrary activity units, p<0.05). Postconditioning also increased the degree of reperfusion achieved as estimated by blush grade (2.44±0.17 vs 1.95±0.27, p<0.05). Therefore, postconditioning in the setting of percutaneous coronary intervention in the cardiology catheterization laboratory was safe and effective [52].

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### REFERENCES

(1) Murry C E, Jennings R B, Reimer K A. Editorial Comment: New insights into potential mechanisms of ischemic preconditioning. Circulation 1991; 84:443-445.
(2) Murry C E, Jennings R B, Reimer K A. Preconditioning with ischemia: a delay of lethal cell injury in ischemic myocardium. Circulation 1986; 74:1124-1136.
(3) Murry C E, Richard V J, Reimer K A, Jennings R B. Ischemic preconditioning slows energy metabolism and delays ultrastructural damage during sustained ischemic episode. Circ Res 1990; 66:913-931.
(4) Reimer K A, Murry C E, Yamasawa I, Hill M L, Jennings R B. Four brief periods of myocardial ischemia cause no cumulative ATP loss or necrosis. Am J Physiol 1986; 251:H1306-H1315.
(5) Downey J M. Ischemia preconditioning. Nature's own cardioprotective intervention. Trends Cardiovasc Med 1992; 2:170-176.
(6) Zhao Z-Q, Nakamura M., Wang N-P, Velez D A, Hewan-Lowe K. O, Guyton R A, Vinten-Johansen J. Dynamic progression of contractile and endothelial dysfunction and infarct extension in the late phase of reperfusion. J Surg Res 2000; 94: 133-144.
(7) Lefer A M, Ma X-L, Weyrich A, Lefer D J. Endothelial dysfunction and neutrophil adherence as critical events in the development of reperfusion injury. Agents Actions Suppl 1993; 41: 127-135.
(8) Davenpeck K L, Gauthier T W, Lefer A M. Inhibition of endothelial-derived nitric oxide promotes P-selectin expression and actions in the rat microcirculation [see comments]. Gastroenterology 1994; 107:1050-1058.
(9) Duilio C, Ambrosio G, Kuppusamy P, DiPaula A, Becker L C, Zweier J L. Neutrophils are primary source of O₂ radicals during reperfusion after prolonged myocardial ischemia. Am J Physiol (Heart Circ Physiol) 2001; 280(6):H2649-H2657.
(10) Hoshida S, Yamashita N, Igarashi J, Nishida M, Hori M, Kamada T et al. Nitric oxide synthase protects the heart against ischemia-reperfusion injury in rabbits. J Pharmacol Exp Ther 1995; 274:413-418.
(11) Buus N H, Bottcher M, Hermansen F, Sander M, Nielsen T T, Mulvany M J. Influence of nitric oxide synthase and adrenergic inhibition on adenosine-induced myocardial hyperemia. Circulation 2001; 104(19):2305-2310.
(12) Barta E, Pechan I, Cornak V, Luknarova O, Rendekova V, Verchovodko P. Protective effect of alpha-tocopherol and L-ascorbic acid against the ischemic-reperfusion injury in patients during open-heart surgery. Bratisl Lek Listy 1991; 92:174-183.
(13) Ooiwa H, Janero D R, Stanley A W H, Downey J M. Examination of 2 Small-Molecule Antiperoxidative Agents in A Rabbit Model of Postischemic Myocardial-Infarction. J Cardiovasc Pharmacol 1991; 17(5):761-767.
(14) Axford-Gately R A, Wilson G J. Myocardial infarct size reduction by single high dose or repeated low dose vitamin E supplementation in rabbits [see comments]. Can J Cardiol 1993; 9:94-98.
(15) Euler D E. Role of oxygen-derived free radicals in canine reperfusion arrhythmias. Am J Physiol 1995; 268:H295-300.
(16) Ambrosio G, Zweier J L, Jacobus W E, Weisfeldt M L, Flaherty J T. Improvement of postischemic myocardial function and metabolism induced by administration of deferoxamine at the time of reflow: the role of iron in the pathogenesis of reperfusion injury. Circulation 1987; 76(4):906-915.
(17) Katoh S, Toyama J, Kodama I, Kamiya K, Akita T, Abe T. Deferoxamine reduces the reperfusion injury in isolated neonatal rabbit hearts after hypothermic preservation. Surg Today 1993; 23:424-429.
(18) Gumina R J, Mizumura T, Beier N, Schelling P, Schultz J J, Gross G J. A new sodium/hydrogen exchange inhibitor, EMD 85131, limits infarct size in dogs when administered before or after coronary artery occlusion. J Pharmacol Exp Ther 1998; 286(1):175-183.
(19) Karmazyn M. Pharmacology and clinical assessment of cariporide for the treatment coronary artery diseases. Expert Opinion on Investigational Drugs 2000; 9(5):1099-1108.
(20) Miura T, Liu Y, Goto M, Tsuchida A, Miki T, Nakano A et al. Mitochondrial ATP-sensitive K+ channels play a role in cardioprotection by Na+-H+ exchange inhibition against ischemia/reperfusion injury. J Am Coll Cardiol 2001; 37(3):957-963.
(21) Linz W, Albus U, Crause P, Jung W, Weichert A, Scholkens B A et al. Dose-dependent reduction of myocardial infarct mass in rabbits by the NHE-1 inhibitor cariporide (HOE 642). Clin Exp Hypertens 1998; 20(7):733-749.
(22) Zeymer U, Suryapranata H, Monassier J P, Opolski G, Davies J, Rasmanis G et al. The Na+/H+ exchange inhibitor eniporide as an adjunct to early reperfusion therapy for acute myocardial infarction—Results of the evaluation of the safety and cardioprotective effects of eniporide in acute myocardial infarction (ESCAMI) trial. J Am Coll Cardiol 2001; 38(6):1644-1650.
(23) Kato K. Haemodynamic and clinical effects of an intravenous potassium channel opener—a review. Eur Heart J 1993; 14:40-47.
(24) Auchampach J A, Gross G J. Reduction in myocardial infarct size by the new potassium channel opener bimakalim. J Cardiovasc Pharmacol 1994; 23:554-561.
(25) Auchampach J A, Maruyama M, Cavero I, Gross G J. Pharmacological evidence for a role of ATP-dependent potassium channels in myocardial stunning. Circulation 1992; 86:311-319.
(26) Mizumura T, Nithipatikom K, Gross G J, Occlusions. Bimakalim, an ATP-sensitive potassium channel opener, mimics the effects of ischemic preconditioning to reduce infarct size, adenosine release, and neutrophil function in dogs. Circulation 1995; 92:1236-1245.
(27) Gross G J, Auchampach J A, Maruyama M, Warltier D C, Pieper G M. Cardioprotective effects of nicorandil. J Cardiovasc Pharmacol 1992; 20 Suppl 3:S22-S28.
(28) Agnelli G, Pascucci C, Cosmi B, Nenci G G. Affects of hirudin and heparin on the binding of new fibrin to the thrombus in t-PA treated rabbits. Thromb Haemost 1991; 66:592-597.
(29) Lam T T, Fu J, Hrynewycz M, Tso M O M. The Effect of Aurintricarboxylic Acid, an Endonuclease Inhibitor, on Ischemia/Reperfusion Damage in Rat Retina. J Ocular Pharmacology & Therapeutics 1995; 11(3):253-259.
(30) Rosenbaum D M, D'Amore J, Llena J, Rybak S, Balkany A, Kessler J A. Pretreatment with intraventricular aurintricarboxylic acid decreases infarct size by inhibiting apoptosis following transient global ischemia in gerbils. Ann Neurol 1998; 43(5):654-660.
(31) Zhao Z-Q, Morris C D, Budde J M, Wang N-P, Muraki S, Guyton R A et al. Inhibition of apoptosis with aurintricarboxylic acid reduces extension of infarction and improves regional contractile dysfunction during reperfusion. Circulation (suppl 1)[104], II-11. 2001 (Abstract).
(32) Balakirev M Y, Zimmer G. Gradual changes in permeability of inner mitochondrial membrane precede the mitochondrial permeability transition. Arch Biochem Biophys 1998; 356(1):46-54.
(33) Griffiths E J, Halestrap A P. Protection by Cyclosporin A of ischemia/reperfusion-induced damage in isolated rat hearts. J Mol Cell Cardiol 1993; 25:1461-1469.
(34) Hausenloy D J, Maddock H L, Baxter G F, Yellon D M. Inhibiting mitochondrial permeability transition pore opening: a new paradigm for myocardial preconditioning? Cardiovasc Res 2002; 55(3):534-543.
(35) Baines C P, Wang L, Cohen M V, Downey J M. Protein tyrosine kinase is downstream of protein kinase C for ischemic preconditioning's anti-infarct effect in the rabbit heart. J Mol Cell Cardiol 1998; 30(2):383-392.
(36) Auchampach J A and Gross G J. Anti-ischaemic actions of potassium channel openers in experimental myocardial ischaemia/reperfusion injury in dogs. Eur Heart J 14: 10-15, 1993.
(37) Axford-Gately R A and Wilson G J. Myocardial infarct size reduction by single high dose or repeated low dose vitamin E supplementation in rabbits [see comments]. Can J Cardiol 9: 94-98, 1993.
(38) Chen H M, Chen M F and Shyr M H. Prostacyclin analogue (OP-2507) attenuates hepatic microcirculatory derangement, energy depletion, and lipid peroxidation in a rat model of reperfusion injury. J Surg Res 80: 333-338, 1998.
(39) Gross G J, Yao Z, Pieper G M and Auchampach J A. The ATP-regulated potassium channel in ischemia-reperfusion injury. Ann N Y Acad Sci 723: 71-81, 1994.
(40) Grover G J, Dzwonczyk S, Parham C S and Sleph P G. The protective effects of cromakalim and pinacidil on reperfusion function and infarct size in isolated perfused rat hearts and anesthetized dogs. Cardiovasc Drugs Ther 4: 465-474, 1990.
(41) Jackson W F, König A, Dambacher T and Busse R. Prostacyclin-induced vasodilation in rabbit heart is mediated by ATP-sensitive potassium channels. Am J Physiol 264: H238-H243, 1993.
(42) Kodani E, Bolli R, Tang X L and Auchampach J A. Protection of IB-MECA against myocardial stunning in conscious rabbits is not mediated by the A1 adenosine receptor. Basic Res Cardiol 96: 487-496, 2001.
(43) Neely C F, DiPierro F V, Kong M, Greelish J P and Gardner T J. A1 adenosine receptor antagonists block ischemia-reperfusion injury of the heart. Circulation 94: II376-II380, 1996.
(44) Neely CF and Keith IM. A1 adenosine receptor antagonists block ischemia-reperfusion injury of the lung. American Journal of Physiology—Lung Cellular & Molecular Physiology 268: L1036-L1046, 1995.
(45) Ockaili R, Salloum F, Hawkins J and Kukreja RC. Sildenafil (Viagra) induces powerful cardioprotective effect via opening of mitochondrial KATP channels in rabbits. Am J Physiol (Heart Circ Physiol) 283: H1263-H1269, 2002.
(46) Schultz J E and Gross G J. Opioids and cardioprotection. Pharmacol Ther 89: 123-137, 2001.
(47) Thourani V H, Brar S S, Kennedy T P, Thornton L R, Watts J A, Ronson R S, Zhao Z-Q, Sturrock A L, Hoidal JR and Vinten-Johansen J. Nonanticoagulant heparin inhibits NF-kB activation and attenuates myocardial reperfusion injury. Am J Physiol 48: H2084-H2093, 2000.
(48) Kin H, Zhao Z-Q, Wang N-P, Halkos M E, Kerendi F, Guyton R A et al. Pharmacological enhancement of post-conditioning (PEP-C) increases myocardial salvage after acute myocardial infarction. Circulation 2005; 112 (Suppl II):II-309 (abstract). (49) Chiari P C, Bienengraeber M W, Pagel P S, Krolikowski J G, Kersten J R, Warltier D C. Isoflurane protects against myocardial infarction during early reperfusion by activation of phosphatidylinositol-3-kinase signal transduction: evidence for anesthetic-induced postconditioning in rabbits. Anesthesiology 2005; 102:102-109.
(50) Halkos M E, Kerendi F, Corvera J S, Wang N-P, Kin H, Payne C S et al. Myocardial protection with postconditioning is not enhanced by ischemic preconditioning. Ann Thorac Surg 2004; 78:961-969.
(51) Zhao Z-Q, Corvera J S, Halkos M E, Kerendi F, Wang N-P, Guyton R A et al. Inhibition of myocardial injury by ischemic postconditioning during reperfusion: comparison with ischemic preconditioning. Am J Physiol (Heart Circ Physiol) 2003; 285:579-588.

(52) Staat P, Rioufol G, Piot C, et al. Postconditioning the human heart. Circulation 2005; 112:2143-2148.

TABLE 1
DEFINITION OF CONCENTRATIONS

NM = NANOMOLAR OR NANOMOLES/LITER OR PICOMOLES/ML
μM = MICROMOLAR OR μMOLES/LITER OR NANOMOLES/ML SOLUTION OR
BLOOD CONCENTRATION
MM = MILLIMOLES/LITER OR μMOLES/ML SOLUTION OR BLOOD
CONCENTRATION
M = MOLES/LITER = MTLLIMOLES/ML SOLUTION OR BLOOD
CONCENTRATION
MG = MILLIGRAMS
MG/KG = MILLIGRAMS/KG BODY WEIGHT, UNLESS SPECIFIED AS MASS OF
ORGAN OR TISSUE
KIU = KALLIKREIN INHIBITORY UNITS
U/KG = UNITS PER KILOGRAM BODY WEIGHT
NOTE: MOLAR UNITS ARE INDICATED FOR FLUID CONCENTRATIONS.
THIS IS IMPORTANT BECAUSE THE DRUGS CAN BE INFUSED TO REGIONS
OF ORGANS, TISSUES AND CELLS, AND THEREFORE DRUG DOSES IN MG
OR MG/KG ARE NOT ACCURATE DESCRIPTIONS, BUT BLOOD/FLUID
CONCENTRATIONS DELIVERED TO THE TARGET TISSUE ARE MORE
ACCURATE.

THERAPEUTIC ADJUNCTS TO ENHANCE THE ORGAN PROTECTIVE
EFFECTS OF POSTCONDITIONING

| 1. | ANTI-INFLAMMATORY AGENTS | |
|---|---|---|
| | a. | Adenosine (5 μg/kg/min-300 μg/kg/min infusion; 0.05 μg/kg-8 μg/kg bolus; 1-12 mg |
| | | iv bolus over 1-2 minutes, repeated 4 times) |

| | |
|---|---|
| a. | A₁ receptor agonist: for example N(6)-(2-phenylisopropyl)-adenosine (R-PIA), |
| | Cyclohexyladenosine (CHA), cyclopentyladenosine (CPA), CCPA (all 1-5 ug/kg |
| | bolus, 0.5 μg/kg/min-30 μg/kg/min infusion or 100 μg/kg i.v.(42) |
| b. | A1 receptor antagonists: L-97-1: 1-10 mg/kg bolus, 1-10 mg/kg/hour(43; 44) |
| c. | A₂ₐ receptor agonist: CGS21680: (0.2 μg/kg/min) AMP579 ([1S-[1a,2b,3b, |
| | 4a(S*)]]-4-[7-[[2-(3-chloro-2-thienyl)-1-methylpropyl]amino]-[(3)H]- |
| | imidazo[4,5-b]pyridyl-3-yl]cyclopentane carboxamide): 15 μg/kg; IV bolus, or |
| | 50 μg/kg; 14 μg/kg bolus + 1.2 μg/kg/minute IV; ATL146e (4-[3-[6-amino-9-(5- |
| | ethylcarbamoyl-3,4-dihydroxy-tetrahydro-furan-2-yl)-9H-purin-2-yl]-prop-2- |
| | ynyl]-cyclohexanecarboxylic acid methyl ester 0.05-50 μg/kg) or 0.06 μg/kg/min. |
| d. | A₃ receptor agonist stimulators: APNEA |
| | (0.50 μg-600 μg/kg/min infusion, or 10-50 nM); |
| | (N(6)-3-iodobenzyladenosine-5′-N-methyluronamide) (IB-MECA) |
| | 100 μg/kg i.v. up to 500 μg/kg.(42) |
| e. | Anti-inflammatory agents |

| | |
|---|---|
| a. | Adhesion molecule antibodies |
| | 1. anti-P-selectin (0.1 μg/kg-10 μg/kg systemically) (8) |
| | 2. anti-L-selectin (0.1 μg/kg-10 μg/kg systemically) |
| | 3. anti-E-selectin (0.1 μg/kg-10 μg/kg systemically) |
| | 4. anti-ICAM-1 (0.1 μg/kg-10 μg/kg systemically) |
| | 5. anti-PECAM (0.1 μg/kg-10 μg/kg systemically) |
| | 6. anti-CD11 or CD18 (i.e. R15.7)(12) (0.1 μg/kg-10 μg/kg systemically) |
| b. | Anti-coagulants with anti-inflammatory effects: heparin and derivatives, |
| | dermatan sulfate and derivatives |

| | |
|---|---|
| 1. | heparin (unfractionated): 50 U/kg (dose for DVT or PE)-100 U/kg; |
| | 18 U/kg/hour infusion; cardiopulmonary bypass use is 300 Units/kg; |
| | intracoronary or other intra-arterial can be equivalent to 5 Units/kg |
| | body weight |
| 2. | heparin (fractionated, Lovenox): 0.5 mg/kg-50 mg/kg body weight; |
| | 30-50 mg subcutaneously every 12 hours for 7-14 days for DVT or |
| | PE; 1 mg/kg subcutaneously every 12 hours for non-Q-wave |
| | myocardial infarction or unstable angina. |
| 3. | dermatan sulfate and derivatives (i.e. intimatan): 0.5 mg/kg-50 mg/kg |
| | bolus; 0.5 mg/kg/hr-100 mg/kg/hour infusion) |
| 4. | desulfated heparin derivatives: These are non-anticoagulating |
| | heparin derivatives (i.e. O-desulfated heparin(47)) used largely for its |
| | anti-inflammatory effects) at 1 mg/kg-40 mg/kg |

| | |
|---|---|
| c. | Non-steroidal anti-inflammatory agents |

| | |
|---|---|
| a. | aspirin: 325-650 mg orally every 4 hours; equivalent to 5-10 mg/kg |
| | body weight; for acute MI 162-325 mg orally given |
| | once, but ranges from 81-325 mg orally for primary prevention |
| | of MI. Higher doses i.e. 2.6-5.4 grams orally are recommended |
| | every 4 hours for arthritis. |
| b. | ibuprofen (300-800 mg are recommended for osteoarthritis, |
| | 400 mg every 4-6 hours for pain; 200-400 mg orally every 4-6 |
| | hours for fever, with a max dose of 1200 mg/day; 3 mg/kg iv; |
| | solutions for experimental intra-arterial use will range from or 1-300 μM |
| | in concentration. |
| c. | N-acetyl cysteine: (5 μg/kg-10 mg/kg); plasma concentrations |
| | as low as 5 mM, and with the suppression being maximal at 40 mM/L |
| | plasma. |
| d. | COX-2 inhibitors: NS-398, a selective COX-2 inhibitor 1-3 mg/kg |
| | iv or up to 25 μM blood or solution concentration; 40 mg |
| | parecoxib, 1000 mg paracetamol; celecoxib (400 mg p.o. |
| | BID; SC58125 [1-[(4-methylsulfonyl) phenyl]-3-trifluoro- |
| | methyl-5-[(4-fluoro)phenyl] pyrazole] (0.1-25 microM blood or |
| | fluid solution) see Bozza PT, Pacheco P, Yu W, Weller PF. |
| | Prostaglandins Leukot Essent Fatty Acids. 2002 Oct; 67(4): 237-44. |

| | |
|---|---|
| 2. | Nitric oxide and NO-donors, regulators of nitric oxide synthase |
| | activity and enzyme levels |

| | |
|---|---|
| a. | L-arginine: 10 μM-50 mM solution or final plasma |
| | concentration; 10 μg/kg-10 mg/kg body weight bolus |
| | depending on systemic application (larger dose) or selective |
| | delivery to target organ (lower dose); 1 μg/kg/min-10 mg/kg/min |
| | infusion)(10) |
| b. | Sodium nitroprusside (Nitropress): 0.3-10 μg/kg/min iv |
| c. | Nitroglycerin: 5-200 μg/min iv; for intra-arterial use, this can |
| | be 0.5 μg/min or less depending on desired target arterial |
| | dilator effect or undesirable systemic vasodilatory effect. |
| d. | Sildenafil (Viagra): 25 mg orally-50 mg; intravenous dose for |
| | reduction of experimental myocardial infarction: 1-3 mg/kg up |
| | to 10 mg/kg i.v. (45) |
| e. | L-NAME: (1 μg/kg-40 μg/kg; 10 ng/kg/min for selected intra- |
| | arterial use)(11; 10) |
| f. | L-NMMA: (1 μg/kg-40 mg/kg; 10 ng/kg/min) |

| | |
|---|---|
| 3. | Cytokine inhibitors and antibodies |

| | |
|---|---|
| a. | TNFα-R1, TNFα antibodies: 0.1 μg/kg/hour-10 mg/kg/h or 10-300 pg/ml |
| | plasma or fluid concentrations) |
| b. | Anti-interleukins (IL-1, IL-6, IL-8) and regulators |
| | (0.1 μg/kg/hour-10 mg/kg/h) |
| c. | Protective interleukin regulators (IL-10) (1 μg/kg-3 mg/kg) |
| | This covers a range of drugs, i.e. adenosine. |

| | |
|---|---|
| 4. | Prostacyclin and analogs |

| | |
|---|---|
| a. | Prostacyclin (10 nM-1 μM(21) fluid or blood concentrations) |
| | or analogs OP-2507 ([15 cis-14-propylcyclohexyl]- |
| | 16,17,18,19,20-pentanor-9-deoxy-9alpha,6-ni-trilo-PGF, methyl |
| | eater) 1 μg/kg/min(38) or (0.1 mg/kg/d) (Hirano T. Nakafusa Y. |
| | Kawano R. Motoyama K. Arima T. Sugitani A. Tanaka M. The |
| | combined use of prostaglandin I2 analogue (OP-2507) and |
| | thromboxane A2 synthetase inhibitor (OKY-046) strongly |
| | inhibits atherosclerosis of aortic allografts in rats. [Journal |
| | Article] Surgery. 129(5): 595-605, 2001 May.) |

| | |
|---|---|
| 5. | Complement inhibitors: pexelizumab a recombinant, single-chain, |
| | anti-C5 monoclonal antibody, intravenous pexelizumab (2.0 mg/kg |
| | bolus plus 0.05 mg/kg per hour for 24 hours (Verrier ED, Shernan SK, |
| | Taylor KM, Van de Werf F, Newman MF, Chen JC, Carrier M, |
| | Haverich A, Malloy KJ, Adams PX, Todaro TG, Mojcik CF, Rollins SA, |
| | Levy JH; PRIMO-CABG Investigators.); C5a complement |
| | inhibitor 18A10, 1 μM-10 μM. |
| 6. | Anti-histamines: benadryl (0.01 μg/kg-0.1 mg/kg; 5-10 mg); 1-10 mg; |
| | cetirizine dihydrochloride (CTZ) and azelastine (AZE) |

| 2. | ANTI-OXIDANTS | |
|---|---|---|
| | a. | Vitamin C: 250 mg iv or intravascular, 4 times a day, to 3 grams orally four times a |
| | | day; (12; 13), Vitamin E: 10-1000 IU/day; 100 mg/kg BW-500 mg alpha- |
| | | tocopherol/kg) (37) beta-carotene 100 microM blood or solution concentration, 10 mg/kg |
| | | BW-120 mg/kg (Combined supplementation of vanadium and beta- |
| | | carotene suppresses placental glutathione S-transferase-positive foci and enhances |
| | | antioxidant functions during the inhibition of diethylnitrosamine-induced rat liver |
| | | carcinogenesis. [Journal Article] Journal of Gastroenterology & Hepatology. |
| | | 19(6): 683-93, 2004 Jun.); vitamin E plus beta-carotene (100 + 10 mg/kg BW, |
| | | respectively); the antioxidant carotenoid astaxanthin (ASX) 100 mg astaxanthin/kg |
| | b. | Flavanoids: (—) Epicatechin (1 mM fluid concentration) |
| | c. | Glutathione (1 uM to 1 mM fluid concentration), or 50 to 100 micromol/(h/kg). |
| | d. | Superoxide dismutase (1 ng/kg-10 mg/kg; 150-1500 U/kg), catalase (1 ng/kg-10 mg/kg; |
| | | 550-5500 U/kg)(15) alone or in combination |
| | e. | Inhibitors of NADPH oxidase or NAD(P)H oxidase (Diphenyl iodonium, 1-500 μM; |
| | | VF244 1-500 μM solution; 0.01-100 mg/kg bolus; 1-100 mg/kg/hour)(9); |
| | | ethyl gallimidate |
| | f. | Allopurinol (0.1 to 100 mM), 30 mg/kg/d; oxypurinol (0.1 to 10 μM) and other |
| | | inhibitors of xanthine oxidase activity |
| | g. | Deferoxamine: (12.5 mg/kg/d)(16; 17) |
| 3. | PROTEASE INHIBITORS | |
| | a. | Serine protease inhibitors (Aprotinin) |

| | |
|---|---|
| a. | Aprotinin: 70 mg/hour iv for control of bleeding; 1,000 KIU/kg-20,000 KIU/kg |
| | body weight; 100-1,000 KIU/min intravascular infusion; for cardiac |
| | surgery: 280 mg loading dose followed b 35-50 mg/h.)1.4 mg = 10,000 KIU) |

| | |
|---|---|
| b. | Protease activated receptor-type 1 (PAR1) antagonists |

| | |
|---|---|
| a. | BMS-200261 (0.1 μM-10 μM bolus, solution concentration for intravascular |
| | use). |
| b. | PAR1Ant1; peptide sequence: trans-cinnamoyl-Phe(pFluoro)- |
| | D(13),L(87)Phe(pGuanidino)-Leu-Arg-Arg-amide (1 mg/kg) |

| | |
|---|---|
| c. | Protease activated receptor-type 2 (PAR2) agonist |

| | |
|---|---|
| a. | SLIGRL-NH₂ (1 mg/kg) |

| | d. | PAR1 antagonist in the presence of postconditioning |
|---|---|---|
| | e. | PAR2 agonist in the presence of postconditioning |
| | f. | Matrix metalloproteinase inhibitor doxycycline: 10-100 μM/L blood or solution |
| | | concentration; 30 mg/kg per day; BB-94 (50 mg/kg, i.p. in mice) - Ref Lee SR, |
| | | Tsuji K, Lee SR, Lo EH.J Neurosci. 2004 Jan 21; 24(3): 671-8. |
| 4. | SODIUM CHANNEL BLOCKERS | |
| | a. | Class I anti-arrhythmic agents (lidocaine, procaine) (10 nM to 1 mM intravascular |
| | | solution; 1-1.5 mg/kg bolus dose; 4 mg loading doses, total 300 mg dose; 0.5 mg/kg |
| | | dose up to 300 mg total dose); |
| | b. | Amiodarone: 1-8 mg/kg for acute MI arrhythmias (higher doses are experimental) |
| 5. | SODIUM/HYDROGEN EXCHANGE (NHE-1) INHIBITORS | |
| | a. | Cariporide: 0.1-15 μM intravascular solution concentration, 3 mg/kg IV |
| | | bolus; 120 mg tid; 1 mg/kg-10 mg/kg; eniporide: 0.5-15 μM intravascular |
| | | solution concentration.(18-22) |
| | b. | SM-20220 (0.5 mg/kg) |
| 6. | K_{ATP} CHANNEL REGULATING AGENTS | |
| | a. | Non-specific openers: aprikalim: 10 μg/kg bolus plus 0.1-10 μg/kg/min(36; 25; |
| | | 39); chromakalim: 0.1 μg/kg/min intracoronary |
| | | infusion(17); nicorandil: 4-12 mg, 100 μg/kg |
| | | bolus with or without 10-25 μg/kg/min(2; 15; 23); pinacidil: 0.09 μg/kg/min |
| | | intracoronary infusion; (40) bimakalim (EMD52692 1-50 μM solution or blood |
| | | concentration, 1-10 μg/kg bolus with or without 0.1 μg/kg/min(15), with or without |
| | | 0.05-0.5 μg/kg/min constant infusion(24; 27; 26)) lemakalim, ER-001533, minoxidil |
| | | sulphate; adenosine 30 μg/kg-2 mg/kg |
| | b. | Sarcolemmal specific openers: P-1075 (Leo Pharmaceutical Products, 1-30 μM in |
| | | fluid or blood concentrations; in vivo dose has not been established yet) |
| | c. | Mitochondrial specific openers: diazoxide: 1-5 mg/kg, or 100 mg oral dose to |
| | | adults; 30 μM fluid or blood concentration. |
| | d. | K_{ATP} channel opener in the presence of a sodium channel blocker |
| | | plus postconditioning |

| | |
|---|---|
| a. | One of the above mentioned K_{ATP} channel openers in concert with lidocaine |
| b. | Adenosine as a K_{ATP} channel opener in concert with lidocaine or other sodium |
| | channel openers |

| 7. | CALCIUM CHANNEL ANTAGONISTS AND REGULATORS | |
|---|---|---|
| | a. | Verapamil 2.5-10 mg i.v.; orally 80-120 mg three times daily; diltiazem 20 mg |
| | | i.v. or 0.25 mg/kg with 25 mg reboluses (0.35 mg/kg); nifedipine: 20-60 mg orally, |
| | | 2-10 mg or 0.5 mg/kg iv. |
| 8. | OPIOIDS | |
| | a. | Enkephalins, proenkephalins, preproenkephalins. Met5-enkephalin, Leu5- |
| | | enkephalin both at 0.125 mg · kg−1 · h−1. |
| | b. | Delta-opioid receptor agonists BW373U86 (1 mg/kg), intracoronary infusion of |
| | | 0.003 mg/kg; TAN-67 at 0.03 mg/min; [D-Pen(2),D-Pen(5)]enkephalin (DpDPE. |
| | c. | kappa-opioid receptor agonists D-Ala2,D-Leu5]enkephalin (DADLE) 1 mg/kg iv; |
| | | pentazocine 5 mg/kg iv |
| | d. | Morphine 1.5-10 mg subcutaneously or intramuscular; (46) 0.3 mg/kg iv with or |
| | | without 0.8-10 mg/hr iv; for MI: 2-5 mg iv or 1 mg/kg iv; |
| | e. | buprenorphine 300 μg every 4-6 hours, can be given in 3 100-μg/kg bo9lus |
| | | infusions); 1-10 μg/kg intravenous or IM dose. |
| 9. | OTHER ANALGESICS | |
| | a. | Fentanyl: 0.02-0.05 mg/kg |
| 10. | REGULATORS OF THROMBOSIS | |
| | a. | Platelet inhibitory agents |

| | |
|---|---|
| a. | platelet activating factor (PAF) inhibitors: WEB 2086 1 mg/kg; |
| b. | Aspirin: 1 mg-1 gm per adult systemic dose; 0.01 μg/kg-10 mg/kg iv |
| c. | GPIIb/IIa inhibitors |

| | |
|---|---|
| 1. | abciximab (c7E3 Fab; abciximab; ReoPro) 0.25 mg/kg and 0.125 μg/kg/min |
| | iv 10-60 minutes before start of procedure. For unstable angina 10 μg/min |
| | iv for 18-24 hours |
| 2. | tirofiban: 0.4 μg/kg × 30 minutes and 0.1 μg/kg/min |
| 3. | eptifibatide (Integralin) (180 μg/kg and 2 μg/kg/min |
| 4. | L-738,167 A single oral 100-microg/kg dose; a single oral 30-microg/kg dose |
| | 30- and 100-microg/kg doses |
| 5. | oral GPIIb/IIIa inhibitors: |

| | |
|---|---|
| d. | GPIb receptor inhibitors |

| | |
|---|---|
| 1. | ATA (aurintricarboxylic acid) 1 mg/kg/h for at least one hour of reperfusion |

| | |
|---|---|
| e. | ADP inhibitors |

| | |
|---|---|
| 1. | Clopidogrel 75 mg orally, ticlopidine (Ticlid) 250 mg orally. |

| | |
|---|---|
| b. | Thrombin inhibitors: |

| | |
|---|---|
| a. | hirudin: 500 units·kg−1·hr−1 |

| | |
|---|---|
| 1. | Desirudin, a recombinant hirudin is given at 0.1-1 mg/kg; bivalirudin |
| | given as a continuous infusion at 2.5 mg/kg/hour, or 0.1 mg/kg bolus |
| | followed by an infusion of 0.25 mg/kg per hour |

| | |
|---|---|
| b. | Lepirudin: 0.4 mg/kg bolus, 0.15 mg/kg/hour |
| c. | Argatroban: 2 μg/kg/min infusion |
| d. | Thrombin regulating agents, prothrombinase regulators (thrombin-anti- |
| | thrombin,) |
| e. | Inhibitors of tissue factor, FVII/FVIIa, X/Xa, such as tissue factor inhibitory |
| | protein: FXa inhibitor ZK-807834 (CI-1031) and DX9065. Factor VIIa inhibitor |
| | recombinant active site-blocked activated factor VII (rFVIIai) from Novo |
| | Nordisk. |
| f. | Tissue plasminogen activator (tPA) 10 mg/kg body weight Thrombolytics i.e. |
| | streptokinase (0.15 MU/h-15 MU/h), urokinase; reteplase |
| g. | Combination of any of the above, especially GPIIb/IIIa inhibitors, heparin and |
| | aspirin) |
| h. | Other anticoagulants: |

| | |
|---|---|
| 1. | dermatan sulphate, initmatan: 1-9 mg/kg, with or without |
| | infusion of 250 μg/kg/hour |
| 2. | desulfated heparin derivatives: 1-30 mg/kg supplemented each 90 minutes. |

| | c. | Thromboxane A2 inhibitors: TXA(2) synthase inhibitor, OKY-046 100 mg/kg or |
|---|---|---|
| | | 10 microM fluid concentration, or dazoxiben at 100 microM fluid concentration, |
| | | and TXA (36) receptor antagonists S-1452 and ONO-3708; 10 microM; |
| | d. | Phosphodiesterase-5 inhibitor: Tadalafil (Cialis); Vardenafil (Lavitra) or Sildenafil |
| | | (Viagra) 25-100 mg orally or 0.05-3 mg/kg up to 10 mg/kg i.v. for a reduction of |
| | | experimental myocardial infarction |
| 11. | METABOLIC ENHANCING AGENTS | |
| | a. | Glucose 0.1 to 5 mM fluid concentration, or 500 ml 10% glucose |
| | b. | Insulin: Glucose-potassium-insulin infusion (500 ml 10% glucose, 20 mmol |
| | | potassium chloride, 16 units of insulin). Intra-arterial solutions can be |
| | | supplemented with 10 IU/L insulin. |
| | c. | Lactate (10 μM to 1 mM in fluid solution); |
| | d. | pyruvate 10 μM to 100 mM, 100 mg/kg i.v. bolus + 10 mg × kg(−1) × min(−1) intra- |
| | | atrial infusion; Dipyruvyl-acetyl-glycerol (DPAG) ester, a pyruvate |
| | | derivative 8.0 mg/kg/min intravenously. |
| | e. | Amino acids (glutamate (100 uM to 20 mM fluid concentration), aspartate (100 uM |
| | | to 20 mM fluid concentration); |
| 12. | BUFFERING AGENTS AND REGULATORS | |
| | a. | Bicarbonate: 0.1 to 5 mM in solution, 250 mg-1 g systemically or 3 mEq/kg i.v.; |
| | b. | Tris-(hydroxymethyl)-aminomethane (tromethamine or THAM), 2.0 ml/kg, i.v. of |
| | | 0.3 M-THAM, 3 mEq/kg tromethamine; 0.1 to 500 mM; histidine (0.1 to 1 mM), |
| | c. | L-carnosine (beta-alanyl-L-histidine) 1-10 μg/kg, i.v. |
| 13. | TEMPERATURE ALTERATIONS | |
| | a. | Hypothermia (deep 0-10° C.; moderate 11-30° C., mild (31-36° C.). Mild hypothermia |
| | | can be used for cath-lab PTCA procedures, while moderate to deep hypothermia |
| | | can be used for bypass and deep hypothermic circulatory arrest procedures. |
| | b. | Normothermia (37-38° C.) |
| 14. | ENDOTHELIN-1 ANTAGONISTS, INHIBITORS AND REGULATORS | |
| | a. | bosentan (Tracleer, Actelion Pharmaceuticals Ltd) a nonselective ETA and ETB |
| | | receptor antagonist 10 mg/kg |
| | b. | Ex. 127, European Patent Application 404 525 A2, Takeda Chemical Ind., 1991), |
| | | CGS 26061 10 microM fluid or blood concentration |
| | c. | SB 209670 6.25 mg × kg(−1) SC b.i.d |
| | d. | BQ-123, a selective ETA receptor antagonist 3 mg/kg bolus injection was followed |
| | | by infusion for 120 min at a rate of 0.1 mg/kg/min |
| 15. | ACE INHIBITORS: | |
| | a. | Enalapril 1 mg/kg |
| 16. | PRECONDITIONING AND PRECONDITIONING (PRETREATMENT) | |
| | MIMETICS: | |
| | a. | Mimetics include adenosine, opioids, bradykinin, NO, opioids |
| | b. | Preconditioning: 5-20 minutes of ischemia (coronary artery occlusion or global |
| | | ischemia) preceding the index ischemia by 5-30 minutes, or up to 72 hours before |
| | | the index ischemia (ischemia producing the infarct) for late preconditioning. |
| 17. | INHIBITORS OF APOPTOSIS | |
| | a. | Selective Caspase inhibitors eg. CAS 1 tetrapeptide inhibitor AC-DEVD-CHO, |
| | | CAS 3 tetrapeptide inhibitor Ac-DEVD-CHO and non-selective caspase inhibitor |
| | | Z-DEVD-FMK. |
| | b. | Endonuclease inhibitors, i.e. Aurintricarboxylic acid (0.1-10 mg/kg or 10-40 μM/mL). |
| 18. | MITOCHONDRIAL PERMEABILITY TRANSITION PORE OPENING | |
| | INHIBITORS | |
| | e.g., cyclosporin A, sanglifehrin A, OR bongkrekic acid, FK506, 10 nM-10 mM | |
| | solution or blood concentration; 0.1-150 mg/kg bolus (32; 33; 34) | |
| 19. | SIGNAL TRANSDUCTION STIMULATORS AND INHIBITORS | |
| | a. | IP-3 kinase (wortmannin) (10 nM-1 mM in blood or fluid solutions), 0.1-5 mg/kg |
| | b. | p-38 kinase stimulators (anisomycin) 1 μg/mL-10 μg/mL(6), 2 to 20 μM blood or |
| | | solution concentration |
| | c. | PKC stimulators (PMA or phorbol 12-myristate 13-acetate) (0.01 nM-10 μM |
| | | blood or fluid concentration)(35) |
| 20. | ANESTHETICS | |
| | a. | Inhalational |

| | |
|---|---|
| a. | Isoflurane (0.01-4%) |
| b. | Sevoflurane (0.01-4%) |
| c. | Halothane (0.01-4%) |

| | b. | Fentanyl (1 μg/kg-100 mg/kg) |
|---|---|---|
| | c. | Morphine (1 μg/kg-500 mg/kg) |
| 21. | STATINS | |
| | 3-hydroxy-3-methylglutaryl coenzyme A reductase inhibitors | |
| | a. | (Lipitor ®, Leschol ®, Zocor ®) (all 1-80 mg orally) |
| | b. | Rosuvastatin ® (0.25 or 1.25 mg/kg) |

These agents can be used alone to supplement postconditioning, or in any combination with postconditioning. For example, cariporide (sodium-hydrogen exchange inhibitor) can be used with adenosine in addition to postconditioning. The drugs can be given before, during or after postconditioning sequences. The drugs can also be supplemented as needed in the post-ischemic period so that either continuous infusions can be given, or multiple separate infusions can be administered at prescribed times for a prescribed duration. For example, 130 μg/kg/min adenosine can be administered systemically after postconditioning for the first hour of reperfusion and then given as boluses or slow infusions at 130 μg/kg at 6, 12, 24 hours post onset of reperfusion.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of preventing injury to an organ or tissue in a subject before, during or after reperfusion following an ischemic event to the organ or tissue, comprising:
a) stopping perfusion of the organ for from about 5 seconds to about 5 minutes;
b) resuming perfusion of the organ for from about 5 seconds to about 5 minutes;
c) repeating steps a) and b) sequentially for from about 2 to about 50 times;
d) allowing uninterrupted perfusion of the organ or tissue; and
e) administering to the subject an effective amount of one or more tissue protective agents in a pharmaceutically acceptable carrier, thereby preventing injury to the organ or tissue in the subject.

2. The method of claim 1, wherein the organ or tissue is heart, brain, eye, kidney, intestine, pancreas, liver, lung or skeletal muscle.

3. The method of claim 1, wherein the subject is a mammal.

4. The method of claim 3, wherein the mammal is a human.

5. The method of claim 1, wherein stopping perfusion is effected by a balloon within a lumen of a blood vessel that supplies blood to the organ or tissue.

6. The method of claim 5, wherein the balloon is inflatable and deflatable.

7. The method of claim 1, wherein stopping perfusion is effected by external compression of a blood vessel that supplies blood to the organ or tissue.

8. The method of claim 1, wherein a tissue protective agent is one or more selected from the group consisting of sodium/hydrogen exchange (NHE-1) inhibitors; anti-inflammatory agents; anti-oxidants; protease inhibitors; sodium channel blockers; K_{ATP} channel regulating agents; calcium channel antagonists and regulators; opioids; regulators of thrombosis; metabolic enhancing agents; buffering agents and regulators; endothelin-1 antagonists, inhibitors and regulators; inhibitors of apoptosis; mitochondrial permeability transition pore opening inhibitors; signal transduction stimulators and inhibitors; anesthetics; and statins.

9. The method of claim 8, wherein the agent is a sodium/hydrogen exchange inhibitor.

10. The method of claim 9, wherein the sodium/hydrogen exchange inhibitor is cariporide or eniporide.

11. The method of claim 10, wherein the dosage of cariporide is 120 mg three times daily.

12. The method of claim 10, wherein the dosage of eniporide is 3 mg/kg.

13. A method of preventing injury to a heart in a subject diagnosed with an ischemic event of the heart, comprising:
a) clearing a lumen of a coronary artery;
b) perfusing the heart for from about 5 seconds to about 5 minutes;
c) stopping perfusion of the heart for from about 5 seconds to about 5 minutes;
d) repeating steps b) and c) sequentially for from about 2 to about 50 times;
e) allowing uninterrupted perfusion of the heart; and
f) administering to the subject an effective amount of one or more tissue protective agents in a pharmaceutically acceptable carrier, thereby preventing injury to the heart in the subject.

14. The method of claim 13, wherein stopping perfusion is effected by a balloon within a lumen of the coronary artery.

15. The method of claim 14, wherein the balloon is inflatable and deflatable.

16. The method of claim 13, wherein stopping perfusion is effected by external compression of the coronary artery.

17. The method of claim 13, wherein a tissue protective agent is one or more selected from the group consisting of sodium/hydrogen exchange (NHE-1) inhibitors; anti-inflammatory agents; anti-oxidants; protease inhibitors; sodium channel blockers; K_{ATP} channel regulating agents; calcium channel antagonists and regulators; opioids;
regulators of thrombosis; metabolic enhancing agents; buffering agents and regulators; endothelin-1 antagonists, inhibitors and regulators; inhibitors of apoptosis; mitochondrial permeability transition pore opening inhibitors; signal transduction stimulators and inhibitors; anesthetics; and statins.

18. The method of claim 17, wherein the agent is a sodium/hydrogen exchange inhibitor.

19. The method of claim 18, wherein the sodium/hydrogen exchange inhibitor is cariporide or eniporide.

20. The method of claim 19, wherein the dosage of cariporide is 120 mg three times daily.

21. The method of claim 19, wherein the dosage of eniporide is 3 mg/kg.

22. A method of preventing injury to an organ or tissue in a subject before, during or after reperfusion following an ischemic event to the organ or tissue, comprising:
a) reducing perfusion of the organ for from about 5 seconds to about 5 minutes;
b) resuming perfusion of the organ for from about 5 seconds to about 5 minutes;
c) repeating steps a) and b) sequentially for from about 2 to about 50 times;
d) allowing uninterrupted perfusion of the organ or tissue; and
e) administering to the subject an effective amount of one or more tissue protective agents in a pharmaceutically acceptable carrier, thereby preventing injury to the organ or tissue in the subject.

23. The method of claim 22, wherein the organ or tissue is heart, brain, eye, kidney, intestine, pancreas, liver, lung or skeletal muscle.

24. The method of claim 22, wherein the subject is a mammal.

25. The method of claim 24, wherein the mammal is a human.

26. The method of claim 22, wherein reducing perfusion is effected by a balloon within a lumen of a blood vessel that supplies blood to the organ or tissue.

27. The method of claim 26, wherein the balloon is inflatable and deflatable.

28. The method of claim 22, wherein reducing perfusion is effected by external compression of a blood vessel that supplies blood to the organ or tissue.

29. The method of claim 22, wherein a tissue protective agent is one or more selected from the group consisting of sodium/hydrogen exchange (NHE-1) inhibitors; anti-inflammatory agents; anti-oxidants; protease inhibitors; sodium channel blockers; K_{ATP} channel regulating agents; calcium channel antagonists and regulators; opioids; regulators of thrombosis; metabolic enhancing agents; buffering agents and regulators; endothelin-1 antagonists, inhibitors and regulators; inhibitors of apoptosis; mitochondrial permeability transition pore opening inhibitors; signal transduction stimulators and inhibitors; anesthetics; and statins.

30. The method of claim 29, wherein the agent is a sodium/hydrogen exchange inhibitor.

31. The method of claim 30, wherein the sodium/hydrogen exchange inhibitor is cariporide or eniporide.

32. The method of claim 31, wherein the dosage of cariporide is 120 mg three times daily.

33. The method of claim 31, wherein the dosage of eniporide is 3 mg/kg.

34. A method of preventing injury to a heart in a subject diagnosed with an ischemic event of the heart, comprising:
a) clearing a lumen of a coronary artery;
b) perfusing the heart for from about 5 seconds to about 5 minutes;
c) reducing perfusion of the heart for from about 5 seconds to about 5 minutes;
d) repeating steps b) and c) sequentially for from about 2 to about 50 times;
e) allowing uninterrupted perfusion of the heart; and
f) administering to the subject an effective amount of one or more tissue protective agents in a pharmaceutically acceptable carrier, thereby preventing injury to the heart in the subject.

35. The method of claim 34, wherein reducing perfusion is effected by a balloon within a lumen of the coronary artery.

36. The method of claim 35, wherein the balloon is inflatable and deflatable.

37. The method of claim 34, wherein reducing perfusion is effected by external compression of the coronary artery.

38. The method of claim 34, wherein a tissue protective agent is one or more selected from the group consisting of sodium/hydrogen exchange (NHE-1) inhibitors; anti-inflammatory agents; anti-oxidants; protease inhibitors; sodium channel blockers; K_{ATP} channel regulating agents; calcium channel antagonists and regulators; opioids; regulators of thrombosis; metabolic enhancing agents; buffering agents and regulators; endothelin-1 antagonists, inhibitors and regulators; inhibitors of apoptosis; mitochondrial permeability transition pore opening inhibitors; signal transduction stimulators and inhibitors; anesthetics; and statins.

39. The method of claim 38, wherein the agent is a sodium/hydrogen exchange inhibitor.

40. The method of claim 39, wherein the sodium/hydrogen exchange inhibitor is cariporide or eniporide.

41. The method of claim 40, wherein the dosage of cariporide is 120 mg three times daily.

42. The method of claim 40, wherein the dosage of eniporide is 3 mg/kg.
